(19) United States
(12) Patent Application Publication
(10) Pub. No.: US 2023/0167189 A1
(43) Pub. Date: Jun. 1, 2023

(54) Treatment of Multiple Sclerosis with Anti-CD52 Antibodies

(71) Applicant: Genzyme Corporation, Cambridge, MA (US)

(72) Inventor: David H. MARGOLIN, Somerville, MA (US)

(73) Assignee: Genzyme Corporation, Cambridge, MA (US)

(21) Appl. No.: 17/930,867

(22) Filed: Sep. 9, 2022

Related U.S. Application Data
(63) Continuation of application No. 16/606,622, filed on Oct. 18, 2019, now abandoned.
(60) Provisional application No. 62/488,630, filed on Apr. 21, 2017.
(60) Provisional application No. 62/575,119, filed on Oct. 20, 2017.
(60) Provisional application No. 62/647,301, filed on Mar. 23, 2018.

(51) Int. Cl. C07K 16/28; A61P 25/28; A61K 31/192; A61K 31/573

(52) U.S. Cl. CPC C07K 16/2893; A61P 25/28; A61K 31/192; A61K 31/573; A61K 9/0019

(57) ABSTRACT

The present invention relates to treatment of relapsing and progressive forms of multiple sclerosis using a humanized anti-human CD52 IgG₁ monoclonal antibody.

### CROSS-REFERENCE TO RELATED APPLICATIONS

This application is a continuation application of U.S. patent application Ser. No. 16/606,622, filed Oct. 18, 2019, which is a national stage application under 35 U.S.C. § 371 of International Patent Application PCT/US2018/028616, filed Apr. 20, 2018, which claims priority from U.S. Provisional Patent Application 62/488,630, filed Apr. 21, 2017; U.S. Provisional Patent Application 62/575,119, filed Oct. 20, 2017; and U.S. Provisional Patent Application 62/647,301, filed Mar. 23, 2018. The disclosures of those applications are incorporated by reference herein in their entirety.

### SEQUENCE LISTING

The instant application contains a Sequence Listing which has been submitted electronically in XML format and is hereby incorporated by reference in its entirety. The electronic copy of the Sequence Listing, created on Sep. 9, 2022, is named 022548_C1031_SL.xml and is 15,606 bytes in size.

### BACKGROUND OF THE INVENTION

CD52 is a glycosylated, glycosylphosphatidylinositol (GPI)-anchored cell surface protein found in abundance (≥500,000 molecules/cell) on a variety of normal and malignant lymphoid cells (e.g., T and B cells). See, e.g., Hale et al., J Biol Regul Homeost Agents 15:386-391 (2001); Huh et al., Blood 92: Abstract 4199 (1998); Elsner et al., Blood 88:4684-4693 (1996); Gilleece et al., Blood 82:807-812 (1993); Rodig et al., Clin Cancer Res 12:7174-7179 (2006); Ginaldi et al., Leuk Res 22:185-191 (1998). CD52 is expressed at lower levels on myeloid cells such as monocytes, macrophages, and dendritic cells, with little expression found on mature natural killer (NK) cells, neutrophils, and hematological stem cells. Id. CD52 is also produced by epithelial cells in the epididymis and duct deferens, and is acquired by sperm during passage through the genital tract (Hale et al., 2001, supra; Domagala et al., Med Sci Monit 7:325-331 (2001)). The exact biological function of CD52 remains unclear but some evidence suggests that it may be involved in T cell migration and co-stimulation (Rowan et al., Int Immunol 7:69-77 (1995); Masuyama et al., J Exp Med 189:979-989 (1999); Watanabe et al., Clin Immunol 120:247-259 (2006)).

Alemtuzumab is a humanized anti-human CD52 monoclonal antibody that exhibits potent in vitro cytotoxic effects (antibody-dependent cell mediated cytotoxicity (ADCC) and complement-dependent cytotoxicity (CDC)) as well as potent lymphocyte-depleting activity in vivo. Alemtuzumab was approved for treating chronic lymphocytic leukemia (marketed as Campath-1H®, Campath®, or MabCampath®). Alemtuzumab also has been approved for treating relapsing forms of multiple sclerosis (MS), including relapsing-remitting MS (RRMS) with active disease (marketed as Lemtrada®). Because of its safety profile, it is generally recommended that Lemtrada® be reserved for patients who have had an inadequate response to previous treatment with other MS drugs.

MS is a chronic, immune-mediated inflammatory and neurodegenerative disease that affects the central nervous system. It is characterized by loss of motor and sensory function resulting from inflammation, demyelination, and axonal injury and loss (Friese et al., Nat Rev Neurol. 10(4):225-38 (2014); Trapp and Nave, Ann Rev Neurosci. 231:247-69 (2008)). MS patients display a wide range of severe clinical symptoms with increased physical disability, fatigue, pain, and cognitive impairment as the disease progresses. MS affects more than two million people worldwide and is at least two to three times more prevalent in women than in men. It has a significant impact on patients' quality of life and shortens patients' life expectancy by five to ten years on average.

A significant unmet clinical need remains in MS treatment despite the commercial availability of a variety of disease-modifying treatments (DMTs). Current DMTs do not address the full spectrum of the MS population and have limitations related to safety and tolerability. Many DMTs have inconvenient dosing regimens and/or modes of administration. The unmet clinical need is especially high for progressive MS. Anti-CD20 antibody ocrelizumab (Ocrevus®) was recently approved in the United States for treating primary progressive MS. Betaferon® (interferon beta-1b) has been approved in the EU to treat secondary progressive MS with active disease. But there remains a need for high-efficacy therapies with more favorable benefit:risk profiles to facilitate an aggressive treatment paradigm for MS of various levels of severity.

### SUMMARY OF THE INVENTION

The present invention provides methods for MS treatment using anti-human CD52 antibody AB1 or a related antibody. The MS treatment achieves surprisingly safe and efficacious results for both relapsing forms and progressive forms of MS.

In some embodiments, the invention provides a method of treating multiple sclerosis (MS) in a patient (e.g., a human patient) in need thereof, comprising administering to the patient a humanized monoclonal anti-human CD52 IgG₁ antibody whose heavy chain complementarity-determining regions (CDR)1-3 and light chain CDR1-3 comprise the amino acid sequences of SEQ ID NOs:5-10, respectively, at a first dose of 12-60 mg, and after an interval of 12 or more months, administering to the patient the antibody at a second dose of 12-60 mg. In some embodiments, the anti-human CD52 antibody comprises a heavy chain variable domain and a light chain variable domain having the amino acid sequences of SEQ ID NOs:3 and 4, respectively. In further embodiments, the antibody comprises, consists of, or consists essentially of a heavy chain and a light chain having the amino acid sequences of SEQ ID NOs:1 and 2, respectively, with or without the C-terminal lysine in the heavy chain.

The patient may have secondary progressive multiple sclerosis (SPMS) (with or without relapses), primary progressive multiple sclerosis (PPMS), progressive relapsing multiple sclerosis (PRMS), or relapsing multiple sclerosis (RMS).

In some embodiments, the anti-CD52 antibody is administered to the patient by intravenous infusion. For example, the anti-CD52 antibody is administered to the patient at a first dose of 60 mg administered to the patient over 1-5 days (e.g., 12 mg/day for 5 days) and a second dose of 36 mg administered to the patient over 1-3 days (e.g., 12 mg/day for 3 days).

In some embodiments, the anti-CD52 antibody is administered to the patient at a first dose of 48 mg and a second dose of 48 mg, each administered to the patient over 1-4 days (e.g., 12 mg/day for 4 days).

In some embodiments, the anti-CD52 antibody is administered to the patient by subcutaneous injection. For example, the anti-CD52 antibody is administered to the patient at a first dose of 60 mg and a second dose of 60 mg. In some embodiments, the anti-CD52 antibody is administered to the patient at a first dose of 60 mg and a second dose of 36 mg. In some embodiments, the anti-CD52 antibody is administered to the patient at a first dose of 36 mg and a second dose of 36 mg. In some embodiments, the anti-CD52 antibody is administered to the patient at a first dose of 48 mg and a second dose of 48 mg. Each dose of the anti-CD52 antibody may be administered to the patient in a single injection (i.e., at a single injection site) or in multiple injections (i.e., at multiple injection sites).

The patient may be medicated with a corticosteroid (e.g., a glucocorticoid such as methylprednisolone), an antihistamine, an antipyretic, or a nonsteroid anti-inflammatory drug (NSAID, e.g., ibuprofen or naproxen) before, during, and/or after administration of the anti-CD52 antibody. For example, the patient may be treated with methylprednisolone, ibuprofen, or naproxen prior to the antibody administration, and optionally treated with one or more of these drugs after the antibody administration. In some embodiments, the patient is treated with 600 mg naproxen per os (PO) twice daily (BID), 64 mg/day methylprednisolone PO X 2 days, or 100 mg methylprednisolone PO prior to the antibody administration.

In some embodiments of the MS treatment methods of the present invention, the patient may be dosed with the anti-CD52 antibodies 12 or more months apart, for example, 12 months apart, 18 months apart, or 24 months apart. In some embodiments, the second dose is the same as or less than the first dose. The patient may be given one or more further doses, in addition to the initial two doses, of the anti-CD52 antibody if he/she displays renewed MS activity or worsening of the disease, or at a certain interval after the last dosing (e.g., 6 months, 48 weeks, 12 months, 18, or 24 months after the last dosing) even in the absence of renewed MS activity or worsening of the disease. The further dose(s) may be 12-60 mg/dose and may be, for example, the same as or less than the previous doses. In some embodiments, the patient is given a fixed dose of 36 mg, 48 mg, or 60 mg of AB1 in each of the first and second doses and in further dose(s) upon display of renewed MS activity or worsening of the disease.

In some embodiments, the present invention provides a method of treating multiple sclerosis (e.g., RMS, SPMS (with or without relapses), PPMS, or PRMS) in a human patient in need thereof, comprising administering to the patient by subcutaneous injection an anti-human CD52 antibody whose heavy chain and light chain comprises the amino acid sequences of SEQ ID NOs:1 and 2, respectively, at a first dose of 60 mg, and at 12 months, administering to the patient by subcutaneous injection the antibody at a second dose of 60 mg. Each of the first and second doses may be administered in a single injection to the patient. The patient may be treated with a corticosteroid, an antihistamine, an antipyretic, or an NSAID PO before the antibody administration, and/or treated with one or more of these drugs after the antibody administration, as described above. In certain embodiments, the patient may be treated with acyclovir and/or methylprednisolone PO before and/or after antibody administration. For example, the patient may be treated with 200 mg of acyclovir twice daily for 28 days beginning on the first day of each antibody treatment course.

The invention also provides use of AB1 or a related anti-CD52 antibody described herein for the manufacture of a medicament for treating MS in a human patient in need thereof in accordance with a treatment method described herein. In some embodiments, the treatment comprises or consists of administration of the antibody at a first dose of 12-60 mg, and after an interval of 12 or more months, administration at a second dose of 12-60 mg. In certain embodiments, the antibody is administered to the patient by subcutaneous injection. In certain embodiments, the antibody is a humanized monoclonal anti-human CD52 IgG₁ antibody whose heavy chain CDR1-3 and light chain CDR1-3 comprise the amino acid sequences of SEQ ID NOs:5-10, respectively.

The invention further provides AB1 or a related anti-CD52 antibody described herein for use in treating MS in a human patient in need thereof in accordance with a treatment method described herein. In some embodiments, the treatment comprises or consists of administration of the antibody at a first dose of 12-60 mg, and after an interval of 12 or more months, administration at a second dose of 12-60 mg. In certain embodiments, the antibody is administered to the patient by subcutaneous injection. In certain embodiments, the antibody is a humanized monoclonal anti-human CD52 IgG₁ antibody whose heavy chain CDR1-3 and light chain CDR1-3 comprise the amino acid sequences of SEQ ID NOs:5-10, respectively.

Also provided in the invention are articles of manufacture, kits, and devices (e.g., a pre-filled, single use syringe or injector) containing a single dose of AB1 or a related anti-CD52 antibody described herein (e.g., 12 mg, 24 mg, 36 mg, 48, mg, or 60 mg), to be used in accordance with a treatment method described herein. In some embodiments, the article of manufacture, kit, or device is for use in treating MS in a human patient in need thereof. In some embodiments, the treatment comprises or consists of administration of the antibody at a first dose of 12-60 mg, and after an interval of 12 or more months, administration at a second dose of 12-60 mg. In particular embodiments, the antibody is administered by subcutaneous injection (e.g., the single dose of the anti-CD52 antibody may be in a container for subcutaneous delivery). Thus, a kit of the invention may comprise, for example, (1) a container that comprises a single dose of 12-60 mg (e.g., 60 mg) of the antibody, wherein the container is for subcutaneous delivery; and (2) a label associated with the container. An article of manufacture of the invention may comprise, for example, a container that comprises a single dose of 12-60 mg (e.g., 60 mg) of the antibody, wherein said container is for subcutaneous delivery. In certain embodiments, the antibody is a humanized monoclonal anti-human CD52 IgG₁ antibody whose heavy chain CDR1-3 and light chain CDR1-3 comprise the amino acid sequences of SEQ ID NOs:5-10, respectively. In particular embodiments, the antibody comprises a heavy chain and a light chain having the amino acid sequences of SEQ ID NOs:1 and 2, respectively.

### BRIEF DESCRIPTION OF THE DRAWINGS

FIG. 1 is a schematic drawing showing a clinical study design for AB1.

FIGS. 2A and 2B are graphs showing the mean serum concentrations of AB1 over time in patients given a single dose of the drug intravenously (IV) (FIG. 2A; 1 mg, 3.5 mg, or 12 mg) or subcutaneously (SC) (FIG. 2B; 12 mg, 36 mg, or 60 mg) in the clinical study.

FIG. 3A is a graph showing the lymphocyte counts in patients given a single IV dose of placebo, or 1 mg, 3.5 mg or 12 mg of AB1 in the clinical study. EOS: end of study. CS: corticosteroid (methylprednisolone). IB: ibuprofen. Premed: pre-medicated (given prior to, or prior to and after, antibody administration).

FIG. 3B is a graph showing the lymphocyte counts in patients given a single SC dose of placebo, or 12 mg, 36 mg or 60 mg of AB1 in the clinical study. EOS: end of study. IB: ibuprofen. Premed: pre-medicated (given prior to, or prior to and after, antibody administration). Prophyl: prophylaxis (given after antibody administration).

FIG. 4A is a graph showing the plasmacytoid dendritic cell (pDC) counts in patients given a single IV dose of placebo, or 1 mg, 3.5 mg or 12 mg of AB1 in the clinical study. EOS: end of study. CS: corticosteroid (methylprednisolone). IB: ibuprofen. Premed: pre-medicated (given prior to, or prior to and after, antibody administration).

FIG. 4B is a graph showing the pDC counts in patients given a single SC dose of placebo, or 12 mg, 36 mg or 60 mg of AB1 in the clinical study. EOS: end of study. IB: ibuprofen. Premed: pre-medicated (given prior to, or prior to and after, antibody administration). Prophyl: prophylaxis (given after antibody administration).

FIG. 5A is a graph showing the natural killer (NK) cell counts in patients given a single IV dose of placebo, or 1 mg, 3.5 mg or 12 mg of AB1 in the clinical study. EOS: end of study. CS: corticosteroid (methylprednisolone). IB: ibuprofen. Premed: pre-medicated (given prior to, or prior to and after, antibody administration).

FIG. 5B is a graph showing the NK cell counts in patients given a single SC dose of placebo, or 12 mg, 36 mg or 60 mg of AB1 in the clinical study. EOS: end of study. IB: ibuprofen. Premed: pre-medicated (given prior to, or prior to and after, antibody administration). Prophyl: prophylaxis (given after antibody administration).

FIG. 6A is a graph showing the CD4⁺ T cell counts in patients given a single IV dose of placebo, or 1 mg, 3.5 mg or 12 mg of AB1 in the clinical study. EOS: end of study. CS: corticosteroid (methylprednisolone). IB: ibuprofen. Premed: pre-medicated (given prior to, or prior to and after, antibody administration).

FIG. 6B is a graph showing the CD4⁺ T cell counts in patients given a single SC dose of placebo, or 12 mg, 36 mg or 60 mg of AB1 in the clinical study. EOS: end of study. IB: ibuprofen. Premed: pre-medicated (given prior to, or prior to and after, antibody administration). Prophyl: prophylaxis (given after antibody administration).

FIG. 7A is a graph showing the CD8⁺ T cell counts in patients given a single IV dose of placebo, or 1 mg, 3.5 mg or 12 mg of AB1 in the clinical study. EOS: end of study. CS: corticosteroid (methylprednisolone). IB: ibuprofen. Premed: pre-medicated (given prior to, or prior to and after, antibody administration).

FIG. 7B is a graph showing the CD8⁺ T cell counts in patients given a single SC dose of placebo, or 12 mg, 36 mg or 60 mg of AB1 in the clinical study. EOS: end of study. IB: ibuprofen. Premed: pre-medicated (given prior to, or prior to and after, antibody administration). Prophyl: prophylaxis (given after antibody administration).

FIG. 8A is a graph showing the CD19⁺ B cell counts in patients given a single IV dose of placebo, or 1 mg, 3.5 mg or 12 mg of AB1 in the clinical study. EOS: end of study. CS: corticosteroid (methylprednisolone). IB: ibuprofen. Premed: pre-medicated (given prior to, or prior to and after, antibody administration).

FIG. 8B is a graph showing the CD19⁺ B cell counts in patients given a single SC dose of placebo, or 12 mg, 36 mg or 60 mg of AB1 in the clinical study. EOS: end of study. IB: ibuprofen. Premed: pre-medicated (given prior to, or prior to and after, antibody administration). Prophyl: prophylaxis (given after antibody administration).

FIG. 9A is a graph showing the percentage of regulatory T (Treg) cells in CD4⁺ T cells during lymphocyte repopulation in patients given a single IV dose of placebo, or 1 mg, 3.5 mg, or 12 mg of AB1 in the clinical study. EOS: end of study. CS: corticosteroid (methylprednisolone). IB: ibuprofen. Premed: pre-medicated (given prior to, or prior to and after, antibody administration).

FIG. 9B is a graph showing the percentage of regulatory T (Treg) cells in CD4⁺ T cells during lymphocyte repopulation in patients given a single SC dose of placebo, or 12 mg, 36 mg, or 60 mg of AB1 in the clinical study. EOS: end of study. IB: ibuprofen. Premed: pre-medicated (given prior to, or prior to and after, antibody administration). Prophy: prophylaxis (given after antibody administration).

FIGS. 10A-10D are graphs showing the levels of IFN-γ (A), IL-6 (B), TNF-α (C), and IL-1β (D) over time in patients given a single IV dose of placebo, or 1 mg, 3.5 mg or 12 mg of AB1 in the clinical study. CS: corticosteroid (methylprednisolone). IB: ibuprofen. Premed: pre-medicated (given prior to, or prior to and after, antibody administration).

FIGS. 11A-11D are graphs showing the levels of IFN-γ (A), IL-6 (B), TNF-α (C), and IL-1β (D) over time in patients given a single SC dose of placebo, or 12 mg, 36 mg, or 60 mg of AB1 in the clinical study. IB: ibuprofen. Premed: pre-medicated (given prior to, or prior to and after, antibody administration). Prophy: prophylaxis (given after antibody administration).

FIG. 12 is a pair of goodness-of-fit plots showing population (left) and individual (right) concentrations of AB1 as predicted by a population pharmacokinetic (popPK) model versus observed concentrations of AB1. The dashed line marks a 1:1 fit.

FIG. 13 is a graph showing median absolute T-cell counts (cells/nL) over time with intravenous (IV) and subcutaneous (SC) administration of AB1 at a dose of 1 mg, 3.5 mg, 12 mg, 36 mg, or 60 mg. FIG. 14 is a pair of goodness-of-fit plots showing population (left) and individual (right) T lymphocyte numbers predicted by a mechanism-based PK/pharmacodynamic (PD) model versus observed T lymphocyte numbers. The dashed line marks a 1:1 fit.

FIG. 15 is a pair of graphs showing the extent of T lymphocyte depletion as predicted by a mechanism-based PK/PD model at month 1 following administration of a single subcutaneous dose of AB1 at 12, 24, 36, 48, or 60 mg. Left: median T cell counts. Right: percentage of median T cell count over baseline T cell count. Dashed line represents median absolute T cell counts at month 1 in alemtuzumab studies CAMMS323 and CAMMS324 (pooled). Triangle/error bar at each dose indicates model-predicted median (5 percentile-95 percentile) of T-cell counts at month 1 following a single dose subcutaneous administration of AB1 in 100 clinical trials with 500 virtual MS patients at each dose in each trial.

FIG. 16 is a graph showing a mechanism-based PK/PD model-predicted median T lymphocyte count following two subcutaneous doses of AB1 at 60 mg per dose. Left dot and right dot represent median absolute T cell counts 1 month after the first and second alemtuzumab treatments, respectively, in alemtuzumab studies CAMMS323 and CAMMS324 (pooled); solid line indicates model-predicted median T-cell counts following two subcutaneous doses of AB1 at 60 mg per dose at an 12 month interval in 100 clinical trials with 500 virtual MS patients in each trial.

### DETAILED DESCRIPTION OF THE INVENTION

The present invention provides safe and efficacious treatment of relapsing and progressive MS with AB1 or a related antibody (such as an antibody (e.g., a humanized IgG₁ antibody) having the same heavy and light chain CDRs, or the same heavy and light chain variable domains, as AB1). AB1 is a humanized anti-human CD52 IgG₁ antibody. A “humanized” antibody refers to an antibody in which the framework region sequences and constant region sequences of the antibody are derived from human sequences. Those framework region and constant region sequences may, in some cases, have been modified relative to the cognate human sequences, e.g., to decrease immunogenicity, increase affinity, and/or increase stability of the antibody.

AB1 has a single N-linked glycosylation site in each heavy chain. The calculated molecular weight of AB1 (excluding carbohydrates) is about 150 kDa. Following binding to cell surface CD52, the antibody can trigger ADCC and CDC of CD52-bearing cells. As most of the CD52-bearing cells in the body are lymphocytes (e.g., T cells and B cells), AB1 or a related antibody is an effective lymphocyte-depleting agent useful in patients who can benefit from lymphocyte depletion. See also WO 2010/132659, the disclosure of which is incorporated by reference herein in its entirety.

The AB1 antibody's heavy chain sequence (SEQ ID NO:1) is shown below, with its variable domain sequence in boldface and italics (SEQ ID NO:3) and its CDR1-3 (SEQ ID NOs:5-7, respectively) in boxes:

(SEQ ID No: 1)

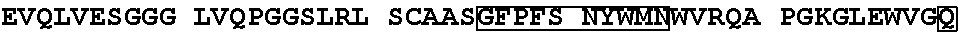

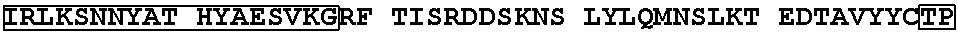

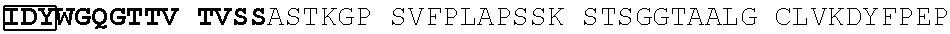

VTVSWNSGAL TSGVHTFPAV LQSSGLYSLS SVVTVPSSSL GTQTYICNVN
HKPSNTKVDK KVEPKSCDKT HTCPPCPAPE LLGGPSVFLF PPKPKDTLMI
SRTPEVTCVV VDVSHEDPEV KFNWYVDGVE VHNAKTKPRE EQYNSTYRVV
SVLTVLHQDW LNGKEYKCKV SNKALPAPIE KTISKAKGQP REPQVYTLPP
SRDELTKNQV SLTCLVKGFY PSDIAVEWES NGQPENNYKT TPPVLDSDGS
FFLYSKLTVD KSRWQQGNVF SCSVMHEALH NHYTQKSLSL SPGK

The AB1 antibody's light chain sequence (SEQ ID NO:2) is shown below, with its variable domain sequence in boldface and italics (SEQ ID NO:4) and its CDR1-3 (SEQ ID NOs:8-10, respectively) in boxes:

(SEQ ID NO: 2)

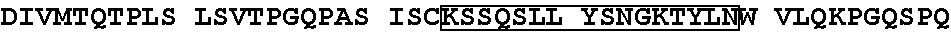

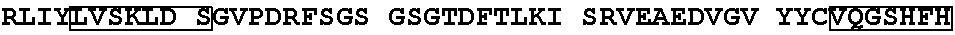

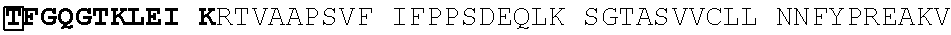

QWKVDNALQS GNSQESVTEQ DSKDSTYSLS STLTLSKADY EKHKVYACEV
THQGLSSPVT KSFNRGEC

AB1 binds to human CD52 at an epitope that is distinct from that of alemtuzumab, with only a partial overlap. Crystallography analysis shows that AB1 binds closer to the N-linked glycosylation site on human CD52 (GQNDTSQTSSPS; SEQ ID NO:11). AB1 contacts residues 5, 7-9, 11, and 12, while alemtuzumab contacts residues 6-12. This difference is thought to result in an epitope binding interaction with different physical characteristics compared to alemtuzumab, with a deeper binding pocket for AB1 and a shallower binding pocket for alemtuzumab.

AB1 and related antibodies used in the present invention can be expressed in, for example, mammalian host cells such as CHO cells, NS0 cells, COS cells, 293 cells, and SP2/0 cells. In some embodiments, the C-terminal lysine of the heavy chain of the antibody is removed. The antibody can be provided, for example, in powder form (e.g., lyophilized form) that is reconstituted in a suitable pharmaceutical solution (e.g., phosphate-buffered saline) before administration to a patient, or in an aqueous pharmaceutical solution. In some embodiments, a pharmaceutical composition comprising the anti-CD52 antibody is provided in an article of manufacture or kit such as one that comprises a container containing the composition and a label associated with the container. The container may be a single use container, such as a single use boule or vial, or a single use, pre-filled syringe or injector (for subcutaneous [SC] delivery). In some embodiments, the container contains a single dose of the anti-CD52 antibody (e.g., AB1), in such an amount as 12 mg, 24 mg, 36 mg, 48 mg, 60 mg, or 90 mg of the antibody, wherein the container may be a vial or a pre-filled syringe or injector. In some embodiments, the article of manufacture or kit comprises one or two of said containers. In certain embodiments, the article of manufacture or kit also comprises a corticosteroid, an antihistamine, an antipyretic, or an NSAID, e.g., for treatment of the patient per os before and/or after administration of the antibody. In particular embodiments, the article of manufacture or kit comprises acyclovir and/or methylprednisolone.

### Types of Multiple Sclerosis

MS, also known as disseminated sclerosis, is a complex disease characterized by considerable heterogeneity in its clinical, pathological, and radiological presentation. It is an autoimmune condition in which the immune system attacks the central nervous system, leading to demyelination (Compston and Coles, Lancet 372(9648):1502-17 (2008)). MS destroys a fatty layer called the myelin sheath that wraps around and electrically insulates nerve fibers. Almost any neurological symptom can appear with the disease, which often progresses to physical and cognitive disability (Compston and Coles, 2008). New symptoms can occur in discrete attacks (relapsing forms), or slowly accumulate over time (progressive forms) (Lublin et al., Neurology 46(4):907-11 (1996)). Between attacks, symptoms may go away completely (remission), but permanent neurological problems often occur, especially as the disease advances (Lublin et al., 1996). Several subtypes, or patterns of progression, have been described, and they are important for prognosis as well as therapeutic decisions. In 1996 the United States National Multiple Sclerosis Society standardized four subtype definitions: relapsing-remitting, secondary progressive, primary progressive, and progressive relapsing (Lublin et al., 1996).

The relapsing-remitting subtype (RRMS) is characterized by unpredictable acute attacks, called exacerbations or relapses, followed by periods of months to years of relative quiet (remission) with no new signs of disease activity. This describes the initial course of most individuals with MS. RRMS is the most heterogeneous and complex phenotype of the disease, characterized by different levels of disease activity and severity, particularly in the early stages. Inflammation is predominant but there is also neurodegeneration. Demyelination occurs during acute relapses lasting days to months, followed by partial or complete recovery during periods of remission where there is no disease activity. RRMS affects about 65-70% of the MS population and tends to progress to secondary progressive MS.

Secondary progressive MS (SPMS) begins with a relapsing-remitting course, but subsequently evolves into progressive neurologic decline between acute attacks without any definite periods of remission, even though occasional relapses, minor remissions or plateaus may appear. Prior to the availability of the approved disease-modifying therapies, data from natural history studies of MS demonstrated that half of RRMS patients would transition to SPMS within 10 years and 90% within 25 years. SPMS affects approximately 20-25% of all people with MS.

The primary progressive subtype (PPMS) is characterized by a gradual but steady progression of disability with no obvious remission after the initial MS symptoms appear (Miller et al., Lancet Neurol 6(10):903-12 (2007)). It is characterized by progression of disability from onset, with occasional temporary minor improvements or plateaus. A small percentage of PPMS patients may experience relapses. Approximately 10% of all individuals with MS have PPMS. The age of onset for the primary progressive subtype is usually later than other subtypes (Miller et al., 2007). Males and females are equally affected.

Progressive relapsing MS (PRMS) is characterized by a steady neurological decline with acute attacks that may or may not be followed by some recovery. This is the least common of all the subtypes described hereinabove.

Cases with non-standard behavior have also been described, sometimes referred to as borderline forms of MS (Fontaine, Rev. Neurol. (Paris) 157 (8-9 Pt 2):929-34 (2001)). These forms include Devic's disease, Balo concentric sclerosis, Schilder's diffuse sclerosis, and Marburg multiple sclerosis (Capello et al., Neurol. Sci. 25 Suppl 4:S361-3 (2004); Hainfellner et al., J. Neurol. Neurosurg. Psychiatr. 55(12):1194-6 (1992)).

The regulatory phrase “relapsing forms of MS” (RMS) generally encompasses both RRMS and SPMS with relapses. The phrase generally refers to three different patient subtypes: RRMS, SPMS with relapses, and a clinically isolated demyelination event with evidence of dissemination of lesions in time and space on the MRI (see, e.g., European Medicines Agency, Committee for Medicinal Products for Human Use's “Guideline on Clinical Investigation of Medicinal Products for the Treatment of Multiple Sclerosis” (Rev. 2, 2015)).

### Treatment of Multiple Sclerosis

The present invention relates to treating various forms of MS with AB1 or a related antibody. The types of MS that can be treated include relapsing MS such as relapsing-remitting MS, primary progressive MS, and secondary progressive MS with or without relapses. MS patients in the context of this invention are those who have been diagnosed as having a form of MS by, for example, the history of symptoms and neurological examination with the help of tests such as magnetic resonance imaging (MRI), spinal taps, evoked potential tests, and laboratory analysis of blood samples.

The present treatment methods can be used as a first line therapy to treat treatment-naïve patients, i.e., those who have not been treated with MS drugs other than corticosteroids. The present treatment methods can also be used to treat patients who have been treated with MS drugs other than corticosteroids, but these patients may have not responded to the previous treatment, or have since experienced worsening of the disease or renewed disease activity.

The treatment methods of the invention will have improved tolerability and more convenient dosing route and regimen. In some embodiments, the invention provides a treatment of RMS or progressive MS with a single dose of AB1 (e.g., 60 mg) by subcutaneous injection, followed by another single dose (e.g., 60 or 36 mg) at 12 months. The current anti-CD52 antibody treatment with alemtuzumab (Lemtrada®) requires five days of IV infusion, followed 12 months after by another three days of IV infusion. Each day of Lemtrada® treatment requires the patient to spend as many as eight hours in a clinical or hospital, which include 4-6 hours of infusion time and time for premedication and post-infusion observation. Thus, presently disclosed embodiments in which a single annual subcutaneous dose is administered will greatly reduce healthcare costs for MS treatment and increase patient comfort and compliance.

Further, the present treatment methods have significantly reduced infusion-associated reactions and thus are advantageous in their safety profiles. Without being bound by theory, the inventors speculate that this advantage is owed to the slow lymphocyte-depleting kinetics of the present treatment and to the resultant lower levels of lymphocyte-lysis induced proinflammatory cytokine release. AB1 also has low immunogenicity in humans. This improved safety profile is not expected to adversely impact the efficacy of the present treatment methods, as potent in vivo T- and B-lymphocyte depletion was observed in patients treated with AB1 and the kinetics of repopulation was observed to be acceptable (see Examples below).

The present treatment methods can be used alone or in combination with other MS drugs. Currently available MS drugs include, for example, oral drugs such as Aubagio® (teriflunomide), Gilenya® (fingolimod), and Tecfidera® (dimethyl fumarate); infusion drugs such as Lemtrada® (alemtuzumab) and Tysabri® (natalizumab); and injectables such as Rebif® (interferon-beta 1a), Plegridy® (pegylated interferon-beta 1a), Copaxone® (glatiramer acetate), and Zinbryta® (daclizumab).

In some embodiments, the anti-CD52 antibody AB1 or a related antibody may be administered to the MS patient intravenously every 3, 6, 12, 18, or 24 or more months. In some embodiments, an IV treatment entails two annual doses given in accordance with the following regimen: (1) 60 mg of AB1 administered to the patient over 1-5 days of daily infusion (e.g., 12 mg/day for 5 days), and 12 months later, 60 mg or 36 mg of AB1 administered to the patient over 1-5 days of daily infusion (e.g., 12 mg/day for 5 or 3 days, respectively); or (2) 48 mg of AB1 administered to the patient over 1-4 days of daily infusion (e.g., 12 mg/day for 4 days), and 12 months later, another 48 mg administered over 1-4 days of daily infusion (e.g., 12 mg/day for 4 days). Each infusion may last 2-4 hours.

Accordingly, in some embodiments, the patient is administered 12 mg/day of AB1 intravenously for 5 days, and 12 months later, administered 12 mg/day of AB1 intravenously for 3 days.

In some embodiments, the patient is administered 12 mg/day of AB1 intravenously for 5 days, and 12 months later, administered 12 mg/day of AB1 intravenously for 5 days.

In some embodiments, the patient is administered 12 mg/day of AB1 intravenously for 4 days, and 12 months later, administered 12 mg/day of AB1 intravenously for 4 days.

In some embodiments, the patient is given a fixed IV dose of 12 mg, 36 mg, 48 mg, or 60 mg of AB1 or a related antibody.

To minimize infusion-associated reactions (IAR; i.e., treatment-emergent adverse events within 24 hours after antibody administration) in IV administration, the patient may be treated with a corticosteroid such as methylprednisolone, an NSAID such as ibuprofen or naproxen, an antipyretic, and/or an antihistamine, by IV or per os (PO), before the antibody administration. If needed, the patient may also be treated with one or more of such medications after the antibody administration. For example, the patient may be pre-treated with 600 mg naproxen PO BID; pre-treated with 64 mg/day methylprednisolone PO X 2 days; pre-treated with 100 mg methylprednisolone PO; pre-treated with 125 mg methylprednisolone IV (30-60 minutes prior to antibody administration); or treated with 400 mg ibuprofen PO prior to and 2 hours after antibody administration.

In some preferred embodiments, the anti-CD52 antibody may be administered to the MS patient subcutaneously (SC) every 3, 6, 12, 18, or 24 months. Exemplary SC treatment entails two annual doses of AB1 given in accordance with one of the following regimens: (1) a single SC dose of 60 mg, and 12 months later, another single SC dose of 60 mg; (2) a single SC dose of 60 mg, and 12 months later, a single SC dose of 36 mg; (3) a single SC dose of 36 mg, and 12 months later, another single SC dose of 36 mg; or (4) a single SC dose of 48 mg, and 12 months later, another single SC dose of 48 mg. The volume for SC injection preferably is small, for example, no larger than 1.2 mL per injection site. Each SC dose may be given to the patient in a single injection or in multiple injections.

Accordingly, in some embodiments, the patient is administered a single SC dose of 60 mg of AB1 in a single injection, and 12 months later, a single SC dose of 60 mg of AB1 in a single injection.

In some embodiments, the patient is administered a single SC dose of 60 mg of AB1 in multiple injections, and 12 months later, a single SC dose of 60 mg of AB1 in multiple injections.

In some embodiments, the patient is administered a single SC dose of 60 mg of AB1 in a single injection, and 12 months later, a single SC dose of 36 mg of AB1 in a single injection.

In some embodiments, the patient is administered a single SC dose of 60 mg of AB1 in multiple injections, and 12 months later, a single SC dose of 36 mg of AB1 in multiple injections.

In some embodiments, the patient is administered a single SC dose of 36 mg of AB1 in a single injection, and 12 months later, a single SC dose of 36 mg of AB1 in a single injection.

In some embodiments, the patient is administered a single SC dose of 36 mg of AB1 in multiple injections, and 12 months later, a single SC dose of 36 mg of AB1 in multiple injections.

In some embodiments, the patient is administered a single SC dose of 48 mg of AB1 in a single injection, and 12 months later, a single SC dose of 48 mg of AB1 in a single injection.

In some embodiments, the patient is administered a single SC dose of 48 mg of AB1 in multiple injections, and 12 months later, a single SC dose of 48 mg of AB1 in multiple injections.

In some embodiments, the patient is given a fixed SC dose of 12 mg, 36 mg, 48 mg, or 60 mg of AB1.

To minimize adverse events in SC administration, the patient may be treated with a corticosteroid such as methylprednisolone, an NSAID such as ibuprofen or naproxen, an antipyretic, and/or an antihistamine, before the antibody administration. If needed, the patient may also be treated with one or more of such medications after the antibody administration. For example, the patient may be pre-treated with 600 mg of naproxen sodium PO BID; pre-treated with 64 mg/day of methylprednisolone PO X 2 days; pre-treated with 100 mg methylprednisolone PO; treated with 400 mg ibuprofen PO prior to and two hours after the antibody administration; treated with 400 mg ibuprofen PO 6 and 9 hours after the antibody administration; or treated with 400 mg ibuprofen PO 4, 8, and 12 hours after the antibody administration.

In some embodiments, the patient may be treated with an antiviral drug such as acyclovir before and/or after the antibody administration. For example, the patient may be treated with 200 mg acyclovir twice daily for 28 days beginning on the first day of each treatment course with the antibody.

After the first two doses of antibody administration, the patient may be given one or more further doses by IV or SC if the patient experiences renewed MS activity or worsening of the disease. For example, re-treatment may be indicated if: (1) within the previous year, the patient has experienced confirmed disability worsening of ≥1 point confirmed over 3 months, or more in patients with a screening EDSS score of <6.0; (2) within the previous year, the patient has experienced confirmed disability worsening of ≥0.5 points confirmed over 3 months, or more in patients with a screening EDSS score of ≥6.0; (3) within the previous year, the patient has experienced one or more relapses; and/or (4) since their last MRI, the patient has accumulated two or more unique lesions on brain or spinal cord MRIs comprising gadolinium-enhancing lesions (e.g., at least 3 mm in any dimension) and/or new or enlarging MRI T2 lesions (e.g., at least 3 mm in any dimension, or showing at least a 3 mm increase). In some cases, the patient is given the one or more further doses at a certain interval after the last dosing (e.g., 6 months, 48 weeks, 12 months, 18, or 24 months after the last dosing) even when he/she has not yet displayed renewed MS activity or worsening of the disease. The further dose(s) may be the same as or less than the previous doses, and may be 12-60 mg/dose. For example, if the patient has been given two initial doses of 60 mg of AB1 SC each, he/she may be given one or more further dose(s) of 36 mg, 48 mg or 60 mg of AB1 SC. In some embodiments, the patient is given a fixed dose of 36 mg, 48 mg, or 60 mg of AB1 IV or SC for each of the first and second dosage courses and for further doses administered upon renewed MS activity or worsening of the disease.

Since an increased risk of serious infections is expected with lymphocyte depletion, patients with known active infections may not be treated with AB1 until the infections are fully controlled.

Further, lymphocyte depletion can sometimes cause secondary autoimmunity. Thus, in some cases, patients who have been treated with the anti-CD52 antibody should be monitored for signs of any secondary autoimmunity and timely treated. Secondary autoimmunity includes, e.g., idiopathic thrombocytopenic purpura (ITP), autoimmune thyroid disease (e.g., Grave's disease), autoimmune cytopenias such as autoimmune neutropenia, autoimmune hemolytic anemia, and autoimmune lymphopenia, and nephropathies including anti-glomerular basement membrane (GBM) disease (Goodpasture's syndrome). Risk minimization activities include laboratory tests conducted at periodic intervals beginning prior to the initial AB1 dose and continuing until up to 48 months, or more as appropriate, after the last administration in order to monitor for early signs of autoimmune diseases. For example, the following blood tests can be performed: (1) complete blood count with differential (prior to treatment initiation and at monthly intervals thereafter); (2) serum creatinine levels (prior to treatment initiation and at monthly intervals thereafter); (3) urinalysis with microscopy (prior to treatment initiation and at monthly intervals thereafter); and (4) test of thyroid function, such as thyroid stimulating hormone level and anti-thyroid peroxidase (prior to treatment initiation and every 3 months thereafter). Further, anti-nuclear antibodies, anti-smooth muscle antibodies, and anti-mitochrondrial antibodies can be measured; in the event anti-nuclear antibodies are detected, additional assays can be performed to measure anti-double-stranded DNA antibodies, anti-ribonucleoprotein antibodies, and anti-La antibodies. Anti-platelet antibodies can be measured to detect autoimmune thrombocytopenia; and a measurement of blood platelet levels may serve to determine if the presence of anti-platelet antibodies is causing a reduction in platelet number.

Additional post-treatment monitoring may be conducted on a patient treated in accordance with the present invention, including, for example, full bloodwork including levels of hepatic enzymes such as alanine aminotransferase, hemoglobin levels and hematocrit measurements, glucose levels, etc.; kidney function; and cardiovascular function.

IV or SC administration of the anti-CD52 antibody in MS patients results in dose-dependent lymphocyte depletion, a desired biological activity of the antibody. Depletion occurs across all lymphocyte subpopulations, including T cells, B cells, NK cells, plasmacytoid dendritic cells (pDCs), and various subsets thereof. Repopulation of CD4⁺ T cells favors regulatory T cells, as shown by studies described in the Examples below. In some embodiments, an absolute count of lymphocytes or lymphocyte subpopulations of the patient decreases by 60-100%, 80-100%, or more than 90% as compared to a baseline absolute count of lymphocytes following an AB1 dosing. In some embodiments, the absolute count of lymphocytes or lymphocyte subpopulations of the patient decreases by greater than 90% between 24 hours and 20 days, between 48 hours and 15 days, between 3 days and 12 days, between 5 days and 10 days, or between 6 days and 8 days following an AB1 dosing. In some embodiments, the patient's absolute T lymphocyte counts remain decreased by greater than 60-100%, 75-100%, 80-100%, or 90-100% 12 months after an AB1 dosing. In some embodiments, T lymphocyte depletion after a second AB1 dosing is similar to T lymphocyte depletion after the first AB1 dosing. In some embodiments, T lymphocyte depletion after a second AB1 dosing is more complete than that observed after a second alemtuzumab treatment which consists of 3 daily IV infusions of 12 mg each (total=36 mg).

The anti-CD52 antibody treatment of the invention will be efficacious in RMS patients. Efficacy can be indicated by reduction in the annual relapse rate (ARR) and/or the time to relapse, and/or a delay in progression of disability as measured over several years such as five years. In some embodiments, the primary clinical endpoints achievable through the treatment of the invention are (1) a 45% or more reduction in annualized relapse rate (ARR) in a population of 450 or more patients assuming ARR in the control group is 0.29 to 0.49 (alpha=0.05, 2-sided); and/or (2) a 35% or more risk reduction on 6-month confirmed disability worsening (CDW) as assessed by EDSS in a population of 900 or more patients assuming a 15% to 20% event rate by year 2 in the control group (alpha=0.05, 2-sided).

The anti-CD52 antibody treatment of the invention will also be efficacious in progressive MS (SPMS and PPMS) patients. Efficacy can be indicated by prevention or delay of the disability progression, such as the 6-month confirmed disability worsening (CDW) by EDSS-Plus Composite measure (EDSS, Timed 25-foot walk (T25FW), 9 Hole-Peg Test (9-HPT)). In some embodiments, the primary clinical endpoint achievable through the treatment of the invention is a 25% or more risk reduction on 6-month CDW as assessed by EDSS-Plus Composite measure in a population of 800 or more patients. In some embodiments, disability progression on EDSS is defined as an increase of ≥1.0 point from the baseline EDSS when the baseline score is 5.5 or less, and ≥0.5 when the baseline score is more than 5.5. In some embodiments, disability progression on T25FW is defined as a worsening of >20% from the baseline score. In some embodiments, disability progression on 9HPT is defined as a worsening of >20% from the baseline score.

Secondary clinical efficacy endpoints include improvements in disability, relapses, MRI-derived parameters, neurological rating scales, measures of cognitive impairment, fatigue scales, ambulatory index, and clinical global impression of change as assessed by patient and physicians, as well as absence of disease activity (e.g., absence of MRI-activity, relapses and progression). For example, secondary clinical endpoints may include, e.g., time to confirmed disability worsening (confirmed over, e.g., at least three months) as assessed by EDSS-Plus Composite measure; total number of new and/or enlarging T2 hyperintense lesions as detected by brain MRI at, e.g., months 6, 12, and 24; change in brain volume as detected by brain MRI, e.g., from month 6 to month 24; proportion of patients with confirmed disability improvement (CDI) confirmed over, e.g., six months; and annualized relapse rate; or any combination of said endpoints.

Still other clinical efficacy endpoints may include, e.g., time to confirmed disability worsening (e.g., 3 or 6 month) as assessed by EDSS; time to onset of confirmed disability worsening (e.g., 3 or 6 month) as assessed by T25FW test; time to onset of confirmed disability worsening (e.g., 3 or 6 month) as assessed by 9HPT; change of EDSS from baseline, e.g., at months 12 and/or 24; change of T25FW test from baseline, e.g., at months 12 and/or 24; change in performance in 9-HPT from baseline to, e.g., months 12 and/or 24; proportion of patients with no evidence of disease activity (NEDA); total number of Gd-enhancing T1 hyperintense lesions as detected by brain MRI at, e.g., months 6, 12, and 24; change in brain volume as detected by brain MRI from baseline to, e.g., month 24; change in total T2 lesion volume as detected by brain MRI from baseline to, e.g., month 24; and patient reported outcomes; or any combination thereof.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention. All publications and other references mentioned herein are incorporated by reference in their entirety. In case of conflict, the present specification, including definitions, will control. Although a number of documents are cited herein, this citation does not constitute an admission that any of these documents forms part of the common general knowledge in the art. Throughout this specification, exemplary embodiments, and claims, the word “comprise,” or variations such as “comprises” or “comprising” will be understood to imply the inclusion of a stated integer or group of integers but not the exclusion of any other integer or group of integers. The materials, methods, and examples are illustrative only and not intended to be limiting.

### EXAMPLES

### Example 1: Anti-CD52 Treatment in Animal Models for Relapsing and Progressive MS

The activity of an anti-mouse CD52 antibody (Turner et al., Journal of Neuroimmunology 285:4-12 (2015)) was evaluated in an animal model for relapsing/progressive MS. Mice were immunized with a PLP peptide (a component of myelin) to induce a phenotype that resembled MS. The mice exhibited a relapsing form of the disease that over time transitioned into progressive disease. Treatment with the anti-CD52 antibody at an early stage of the disease resulted in a significant decrease in the clinical scores of the mice and this result was sustained throughout the remainder of the experiment. Treatment at a later stage when the mice were beginning to enter the progressive stage of the disease also resulted in a significant decrease in the clinical score compared to vehicle control. Taken together, these data suggest that targeting CD52 in either the relapsing or the progressive stage of the disease will result in a significant decrease in symptoms of the disease.

### Example 2: Nonclinical Pharmacology and Safety Studies

Nonclinical pharmacological studies of AB1 were carried out in human CD52 transgenic mice. This animal model was created using the outbred CD-1 mouse strain and the transgene was placed under the control of a human CD52 promoter. The mice displayed a distribution pattern and expression level of human CD52 similar to that observed in humans. Lymphocyte depletion following administration of AB1 was evaluated by flow cytometry analysis where T lymphocytes were identified by CD3 expression and B lymphocytes were identified by CD19 expression. Subcutaneous (SC) and intravenous (IV) administration of AB1 to the transgenic mice resulted in dose-dependent lymphocyte depletion accompanied by a modest and transient increase in serum cytokines. Repopulation of lymphocytes occurred over time. B lymphocytes repopulated faster than T lymphocytes. The lowest dose associated with minimal pharmacological activity (lymphocyte depletion) was 0.05 mg/kg for IV and 0.5 mg/kg for SC.

Single dose pharmacokinetics of AB1 were evaluated following either IV or SC administration in the huCD52 transgenic mouse model. The pharmacokinetic profile for IV- and SC-administered AB1 was consistent with a one-compartment model at all doses tested. The terminal elimination half-life (t_{1/2}) for AB1 remained fairly consistent over the dose range tested in this study: 56.3 f 16.7 hours for 0.5 mg/kg AB1 administered SC and 57.0 f 18.5 hours for 0.5 mg/kg AB1 administered IV.

Safety studies done in the transgenic mice showed that the exposure ratios at the 6-month NOAEL (no-observed-adverse-effect-level) of Cmax in the transgenic mice (68.6 f 37.8 μg/mL and 48.2 f 44.3 μg/mL in males and females, respectively) to human single dose exposure at 60 mg (Cmax 4.01 μg/mL) were 17- and 12-fold.

### Example 3: Cytokine Response in an In Vitro Human Whole Blood Assay

An in vitro human whole blood assay was developed to compare the cytokine response in blood to AB1 to the cytokine response to alemtuzumab. Whole blood from six donors was tested with each of six concentrations of either antibody (0.01, 0.075, 0.5, 1, 5, and 50 μg/mL), and three cytokines were measured (TNF-α, IFN-γ, and IL-6). Statistically significant differences between the two antibodies were observed at the peak maximal response in all donors for all cytokines. At peak response, AB1 yielded 8-10 fold lower cytokine response for IFN-γ, and 5-fold lower response for TNF-α. The data demonstrate that AB1 results in less proinflammatory cytokine release in vitro compared with alemtuzumab, which may be attributable to altered kinetics of cell depletion observed with AB1 as compared with alemtuzumab, and which is expected to translate to improved tolerability in patients.

### Example 4: AB1 Clinical Study

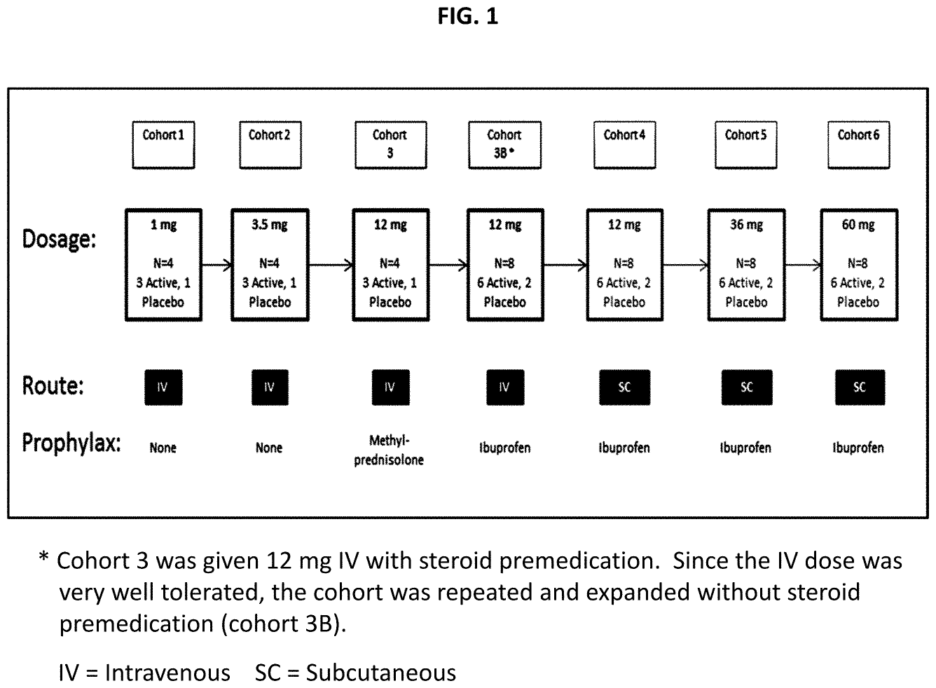

AB1 was investigated as an MS therapeutic in a randomized, double-blind, placebo-controlled clinical study. Men and women aged 18-65 years with progressive multiple sclerosis (including PPMS, SPMS, and progressive relapsing MS patients) were given a particular dose from a range of sequential ascending single IV or SC doses of AB1. The study duration was up to 8 weeks, with 4 weeks of screening and 4 weeks of follow-up after each dose/treatment. The primary endpoint of the study was adverse event (AE) incidence. IARs were defined as treatment-emergent AEs occurring from the time of IV infusion or SC injection until 24 hours after the infusion or injection. The secondary endpoints included lymphocyte counts (including pharmacodynamics effects on innate immune cells (plastmacytoid dendritic cells and natural killer cells) and adaptive immune cells (CD4⁺ and CD8⁺ cells, CD4⁺ Treg cells, and CD19⁺ B cells)). FIG. 1 illustrates the study design.

In this study, a total of 44 patients were randomized into 7 cohorts. The first four cohorts received either placebo or a particular dose of AB1 from a sequential ascending single dose range (FIG. 1). Three dose levels of AB1 (1 mg, 3.5 mg, and 12 mg) were given IV. For the highest IV dose (12 mg; cohort 3 or 3B; FIG. 1), patients were given with either corticosteroid methylprednisolone (125 mg by IV, 30-60 min prior; cohort 3) or ibuprofen (400 mg orally, prior to and two hours after antibody administration; cohort 3B) to minimize IARs.

After review of IV data, SC administration was begun in another three cohorts of patients. The first cohort of SC patients received a SC dose of 12 mg of AB1; these patients also were given 400 mg of ibuprofen orally prior to and 2 hours after the AB1 administration. The second cohort of patients received a SC dose of 36 mg of AB1; these patients were given 400 mg of ibuprofen orally 6 and 9 hours after the AB1 administration. The third cohort of patients received a SC dose of 60 mg of AB; these patients also were given 400 mg of ibuprofen orally 4, 8 and 12 hours after the AB1 administration. AB1 was provided in an aqueous liquid solution at 10 mg/ml. Patients receiving 36 mg or 60 mg of AB1 were given the single SC dose at multiple injection sites (3 or 5 injections of 12 mg of antibody in 1.2 ml).

The demographic characteristics at baseline were similar across all cohorts with the exception of the median time since first diagnosis being higher in the IV cohorts (17.0 years) than in the SC cohorts (5.4 years). Overall, for the IV cohorts, the mean age of population was 54 years (SD: 6.3, range across cohorts was 38 to 64 years), 55.0% of patients were female, all patients were Caucasian (100%), mean body mass index (BMI) was 25.80 kg/m² (SD: 5.07, range across cohorts was 17.6 to 38.2 kg/m²) and mean Expanded Disability Status Scale (EDSS) score was 5.6 (SD: 1.7). Overall, for the SC cohorts, the mean age of population was 50 years (SD: 9.4, range across cohorts was 21 to 61 years), 50.0% of patients were female, all patients were Caucasian (100%), mean BMI was 25.64 kg/m² (SD: 3.08, range across cohorts was 19.2 to 29.5 kg/m²) and mean EDSS score was 5.6 (SD: 1.3).

A. Treatment-Emergent Adverse Events

No deaths, or severe or serious AEs, i.e., serious treatment-emergent adverse events (TEAE), or grade 3 or higher AEs, were reported with AB1 treatment. For IV treatment, the incidence of TEAEs and severity of the events (excluding 12 mg IV with corticosteroid (CS) premedication) revealed no clear relationship with increasing dose. The most common TEAEs following IV administration were headache (9/15 AB1-treated patients vs. 4/5 placebo patients), nausea (6/15 AB1-treated patients vs. 0/5 placebo patients), and body temperature increase (6/15 AB1-treated patients vs. 0/5 placebo patients). Overall, the number of patients reporting IARs following IV administration was 12/15 patients [80.0%] in AB1 groups reporting 59 events and 3/5 patients [60.0%] in the placebo group reporting 3 events. The highest severity IAR reported in AB1 IV groups was grade 2 with 9/15 patients (60%) reporting 18 events. TEAEs of special interest (AESI) were reported in 3 of the IV patients: 1 patient in the 3.5 mg AB1 IV group with alanine aminotransferase increase (4.25× upper limit of normal [ULN] on Day 1) of moderate intensity, 1 patient in the 12 mg AB1 IV group with thrombocytopenia (86×109/L on Day 3) of mild intensity, and 1 patient in the 12 mg AB1 IV group with both thrombocytopenia (89×109/L on Day 3) of mild intensity and alanine aminotransferase increase (3.21×ULN on Day 7) of moderate intensity. All abnormal lab values were in normal range at the time of EOS except one platelet count which was close to the lower limit of normal (LLN).

For SC administration, all patients, including placebo patients, reported at least 1 TEAE. With regard to severity, the grade 2/grade 1 ratios observed in 12, 36, and 60 mg SC groups were 0.20, 0.28, and 0.21, respectively. IARs associated with SC administration occurred in 89% (16/18) of AB1 patients and 83% (5/6) of SC placebo patients. The most common TEAEs following SC administration were injection site erythema (15/18 AB1-treated patients vs. 1/6 placebo patient), increased body temperature (14/18 AB1-treated patients vs. 0/6 placebo patients), headache (13/18 AB1-treated patients vs. 2/6 placebo patients), asthenia (8/18 AB1-treated patients vs. 0/6 placebo patients), and injection site edema (7/18 AB1-treated patients vs. 0/6 placebo patients). AESIs were reported in 1 patient in the 60 mg AB1 SC group, who had hepatic enzyme increased (3.95XULN on Day 2) but recovered within 5 days.

B. Pharmacokinetic Results

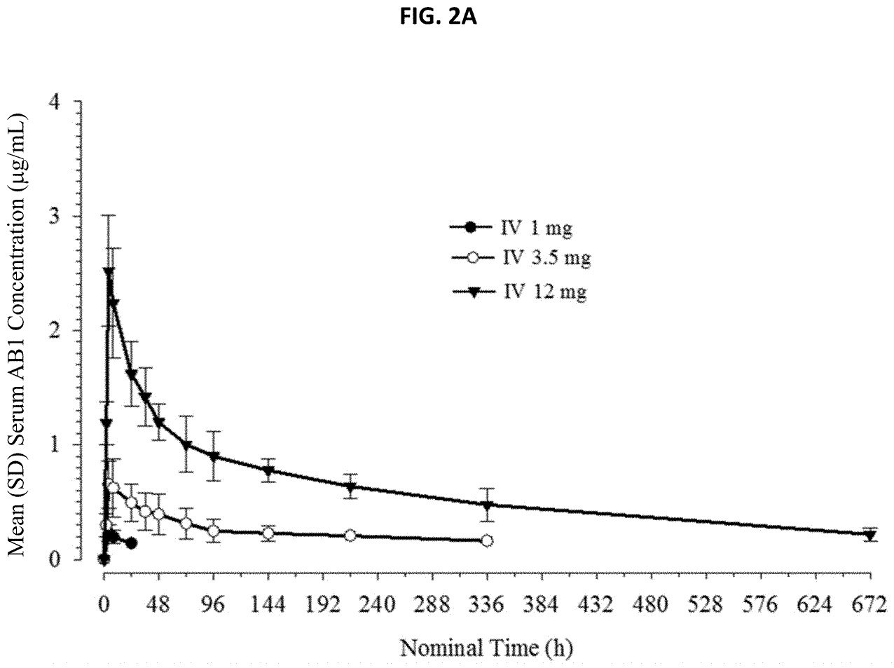

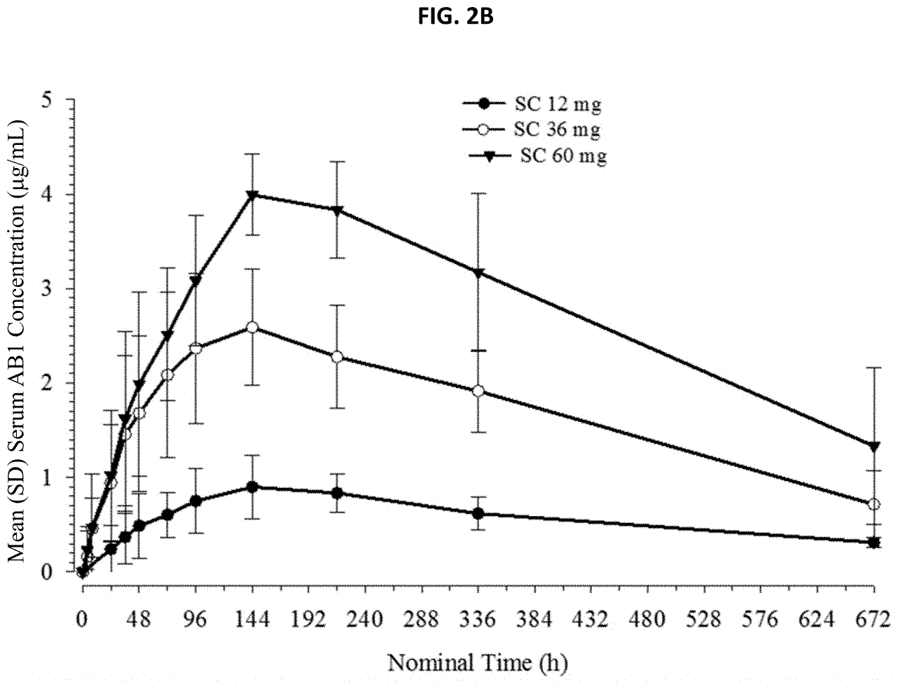

FIG. 2A shows the pharmacokinetics (PK) of AB1 in patients given the antibody by IV (1 mg, 3.5 mg, or 12 mg). Maximal AB1 serum concentrations were mostly observed at the end of infusion, after which they appeared to decline biexponentially. The terminal phase could not be characterized at the low dose of 1 mg. After the 12 mg dose, the mean terminal half-life associated with the terminal slope (t_{1/2z}) was approximately 11 days; the mean total body clearance after infusion (CL) was 27.6 mL/h; and the mean volume of distribution at steady state after single IV infusion dose (Vₛₛ) was 8.64 L. For a 12-fold increase in IV dose from 1 mg to 12 mg, mean maximum serum concentration observed (Cₘₐₓ) increased by 11.2 fold, while the mean area under the serum concentration versus time curve calculated from time zero to the real time corresponding to the last concentration above the limit of quantification (AUCₗₐₛₜ) increased by 167 fold. For a 3.43-fold increase in IV dose from 3.5 mg to 12 mg, mean Cₘₐₓ increased by 3.70 fold, while the mean AUCₗₐₛₜ increased by 5.2 fold.

FIG. 2B shows the pharmacokinetics of AB1 in patients administered the antibody by SC. After a single SC dose, AB1 was absorbed with a median time to reach Cₘₐₓ (tₘₐₓ) of 6.0 to 7.5 days and the mean apparent t_{1/2z} was approximately 13 days (308-315 h). Table 1 below shows the pharmacokinetic parameters of AB1 administered subcutaneously. Mean serum AB1 Cₘₐₓ and area under the serum concentration versus time curve extrapolated to infinity (AUC) values increased approximately proportionally from 12 mg to 36 mg, and increased less than proportionally from 36 mg to 60 mg. For a 5-fold increase in SC dose from 12 mg to 60 mg, mean Cₘₐₓ increased by 4.28 fold, while the mean AUCₗₐₛₜ increased by 4.69 fold. The bioavailability was approximately 100% at the SC doses of 12 mg and 36 mg and approximately 82% at the SC dose of 60 mg.

TABLE 1
Pharmacokinetic Parameters of AB1 Administered Subcutaneously

| | Mean ± SD (Geometric Mean) | | |
|---|---|---|---|
| | [CV %] of AB1 Parameters | | |
| | SC 12 mg | SC 36 mg | SC 60 mg |
| N | 6 | 6 | 6 |
| Cₘₐₓ (μg/mL) | 0.938 ± 0.300 | 2.71 ± 0.621 | 4.01 ± 0.409 |
| tₘₐₓ^{a}(h) | 145.24 (139.75- | 144.41 (96.00- | 181.82 (145.83- |
| | 263.52) | 216.93) | 217.77) |
| t_{1/2z} (h) | 308 ± 104 | 315 ± 117 | 312 ± 130 |
| AUCₗₐₛₜ (μg · h/mL) | 369 ± 105 | 937 ± 348 | 1730 ± 526 |
| AUC (μg · h/mL) | 553 ± 144 | 1510 ± 368 | 1900 ± 266 |
| CL/F (mL/h) | 22.9 ± 7.06 | 25.0 ± 6.89 | 321 ± 4.67 |
| Vₛₛ/F (mL) | NC ± NC | NC ± NC | NC ± NC |
| F (%) | 119 | 108 | 81.5 |

^{a}Median (Min-Max)
NC: not calculated

The mean Cₘₐₓ values at the 12-, 36-, and 60-mg SC doses were 36.9%, 107% and 158% of that at the 12-mg IV dose, respectively, indicating that mean Cₘₐₓ of the 36-mg SC dose is similar to the mean Cₘₐₓ of the 12-mg IV dose (4-hour infusion).

C. Pharmacodynamic Results

Lymphocyte depletion is the principal desired pharmacodynamic (PD) effect of AB1. Dose-dependent lymphocyte depletion was observed in both the IV and SC groups, confirming the desired biological activity of AB1. The maximal extent of depletion was dose-related and very substantial in all AB1 dose groups, with mean absolute lymphocyte count decreases from baseline by more than 90% in 12-mg IV (97.5%), 36-mg SC (92.2%), and 60-mg SC (95.4%). Lymphocyte depletion was incomplete in some patients at 12-mg SC, and was delayed in the 36-mg cohort relative to the 60-mg cohort. Lymphocyte recovery began earlier in lower dose groups that showed less complete depletion. The mean absolute lymphocyte count decrease from baseline on day 15 (D15) was still more than 90% in the 60-mg SC group, and over 80% in the 12-mg IV and 36-mg SC groups. The mean absolute lymphocyte count decrease from baseline on D29 was still more than 80% in the 60-mg SC and 12-mg IV groups and close to 80% in the 36-mg SC group.

(i) IV Administration

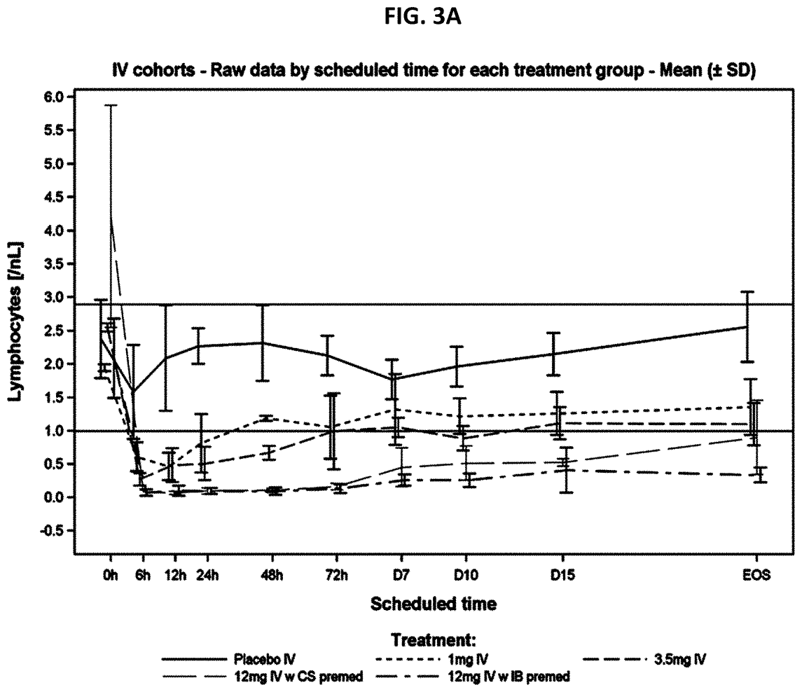

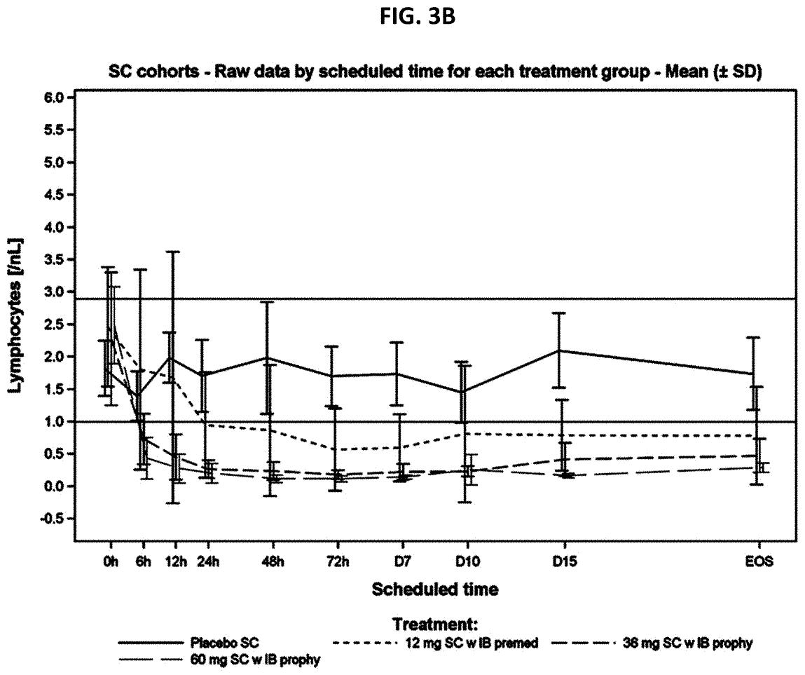

Dose-dependent lymphocyte depletion was observed in all AB1 IV groups as shown in FIG. 3A. In the lowest IV dose group (1 mg; n=3), the mean lymphocyte count was reduced from 1.945/nL at baseline to a nadir of 0.464/nL at 12 hours post treatment. The lymphocyte count was still below baseline at EOS (1.356/nL). In the highest AB1 IV dose group (12 mg; n=9), the mean count was reduced from 2.791/nL at baseline to a nadir of 0.069/nL at 6 hours post treatment, and remained substantially below baseline at EOS (0.520/nL). Depletion across all lymphocyte subpopulations, including T cells, B cells, NK cells, and various subsets thereof, was observed in all groups receiving AB1 IV.

Lymphocyte depletion was dose-dependent. All lymphocyte subsets showed generally similar temporal profiles. The nadir in IV groups was consistently seen within 6 to 12 hours post treatment, with subsequent gradual and only partial recovery of cell counts through EOS in most cases. Some patients in lower-dose groups (<12 mg) fully recovered to baseline values for B cells and NK cells.

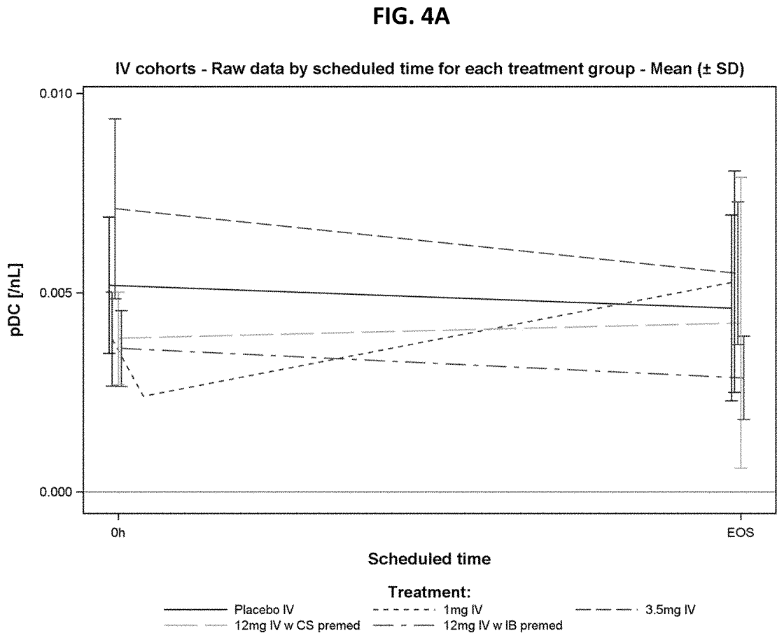

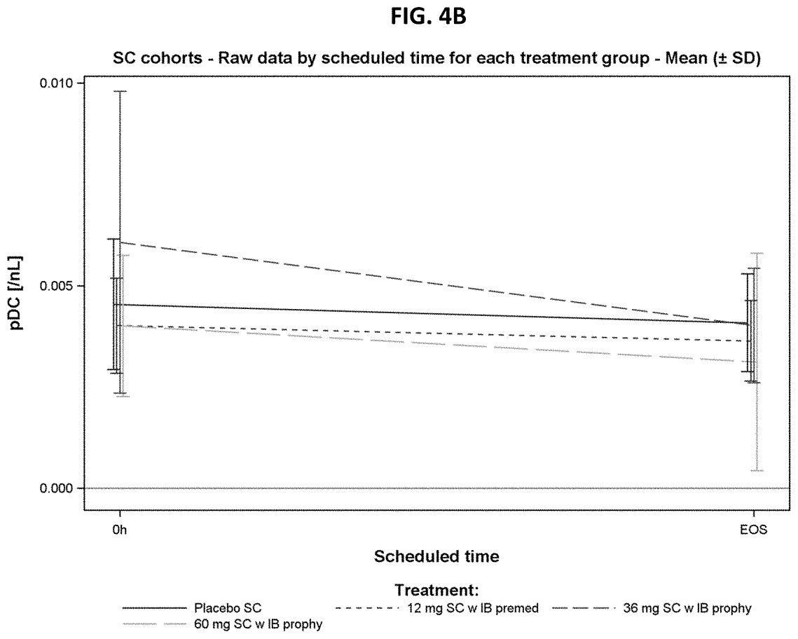

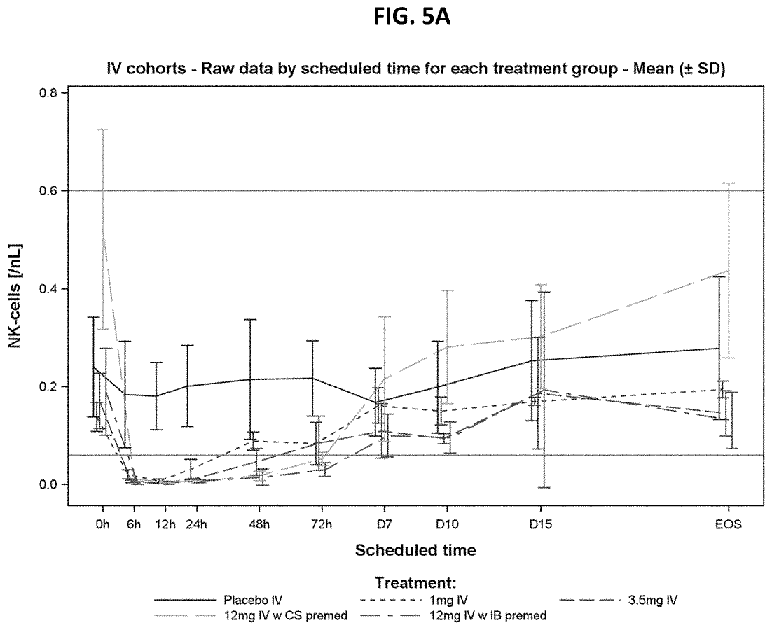

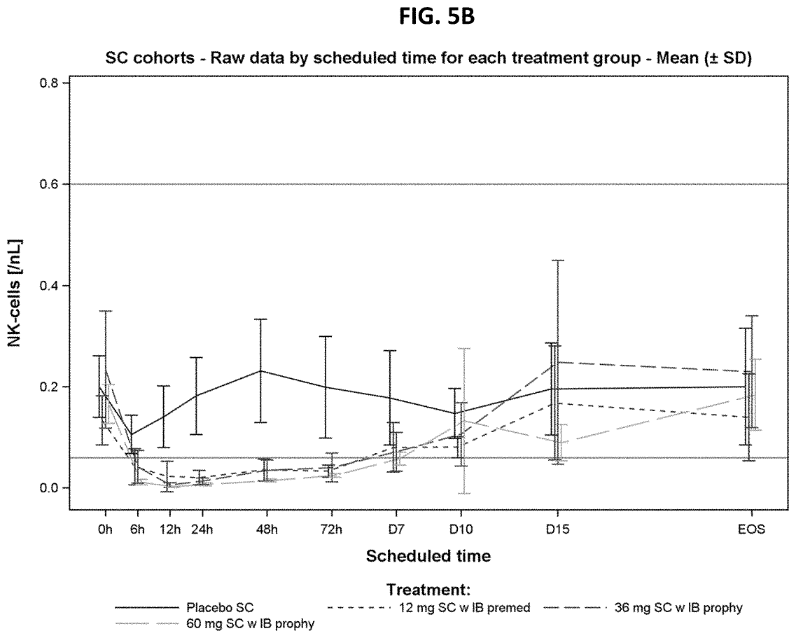

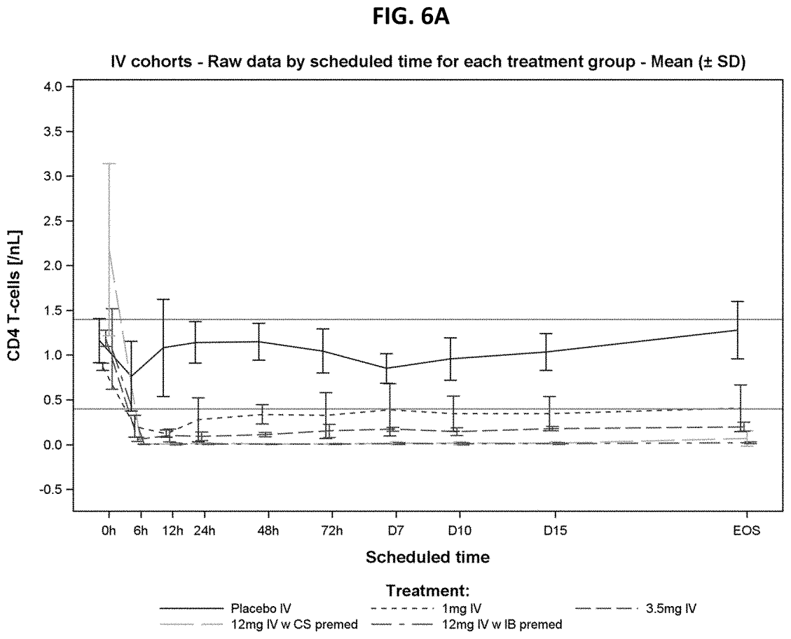

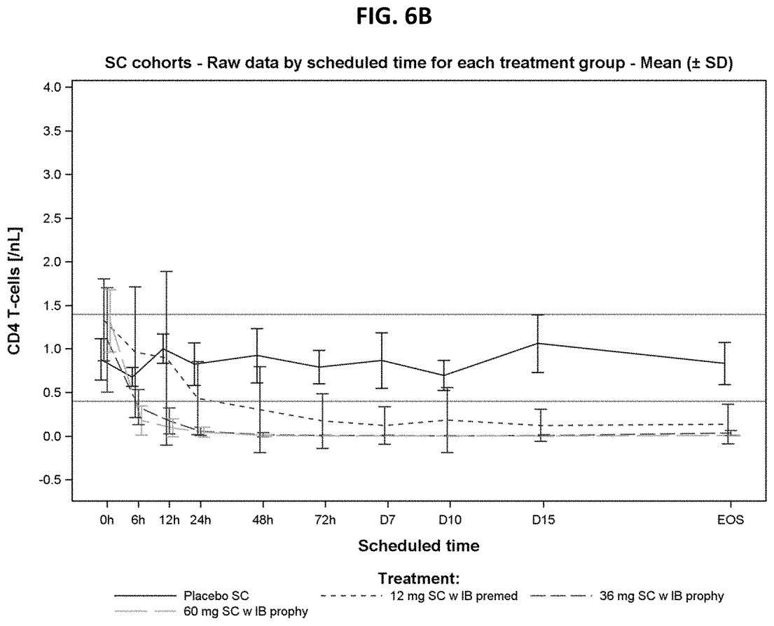

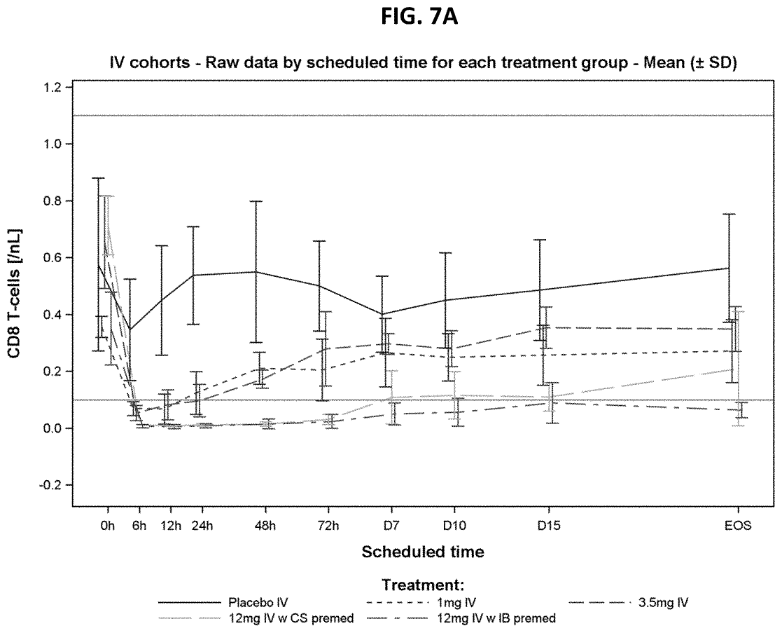

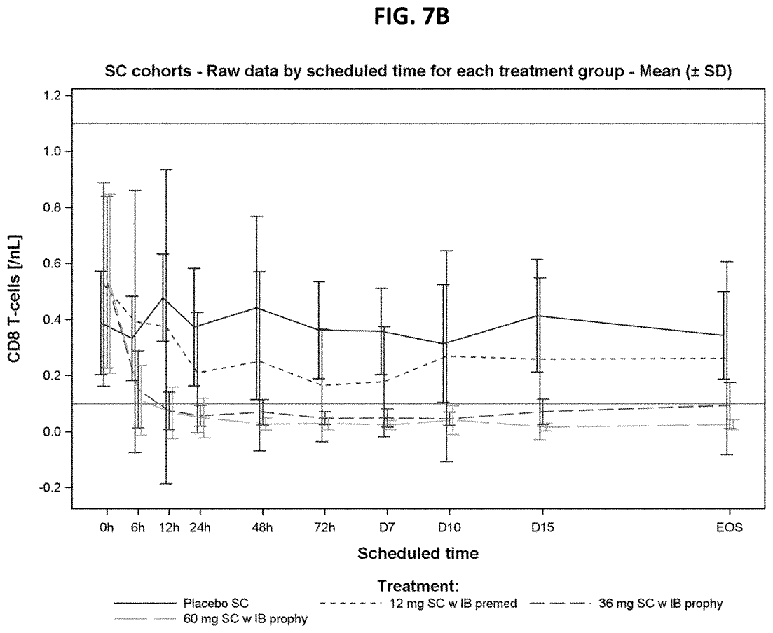

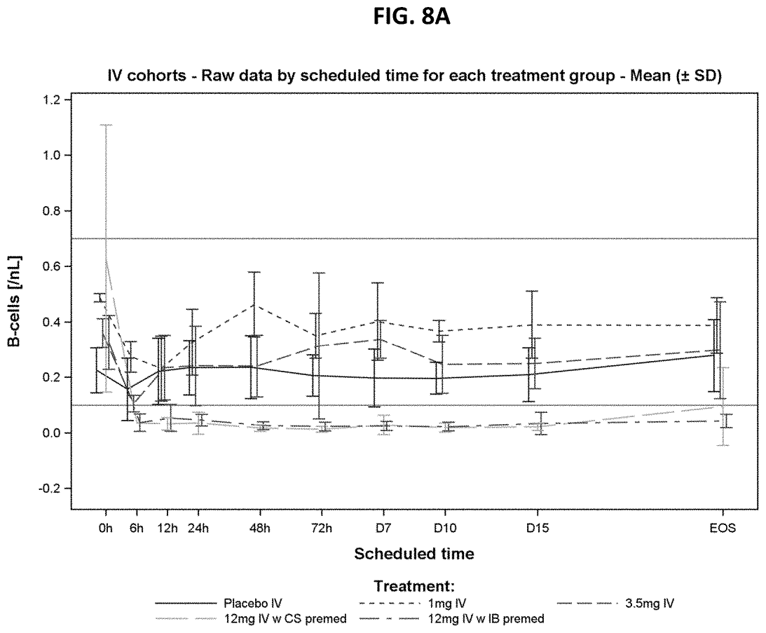

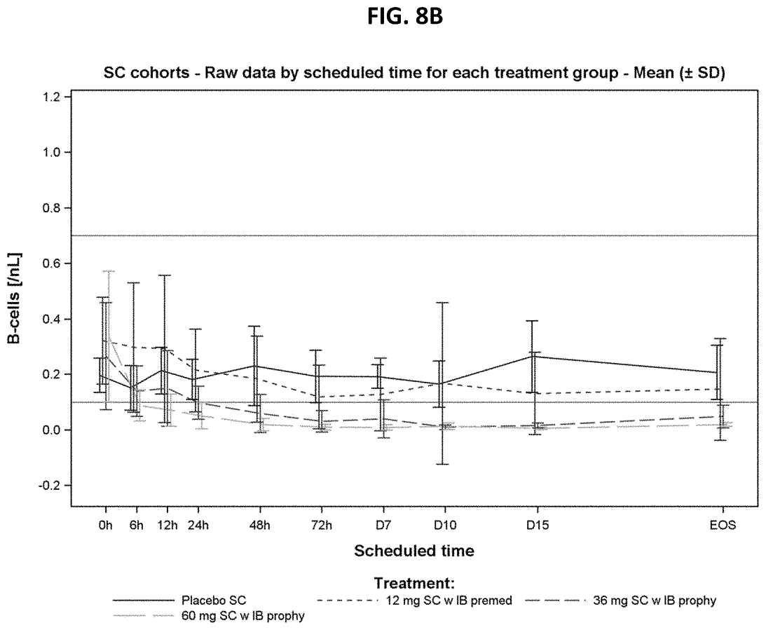

pDC counts remained stable following IV AB1 and were comparable to placebo (FIG. 4A). NK cell counts showed marked depletion following IV AB1 versus placebo, but recovered by Day 10 (FIG. 5A). CD4⁺, CD8⁺, and CD19⁺ lymphocyte counts decreased dose-dependently and more rapidly with IV (six hours) versus SC (12-24 hours for CD4⁺ and CD8⁺ T lymphocytes, and 48-72 hours for CD19⁺ lymphocytes) administration (FIGS. 6A, 7A, and 8A). Mean counts were >90% lower than baseline at the end of study in the highest IV dose cohort.

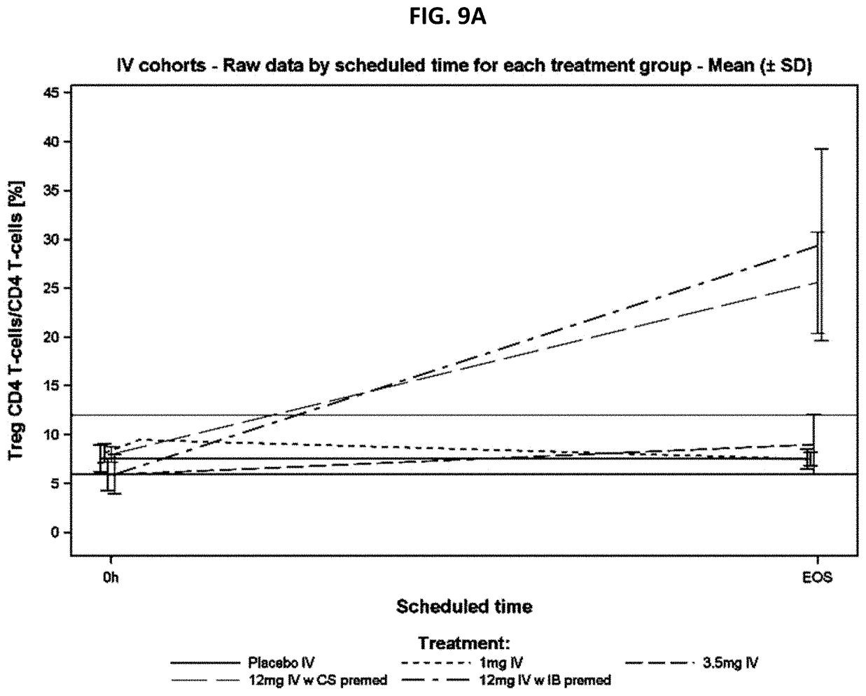

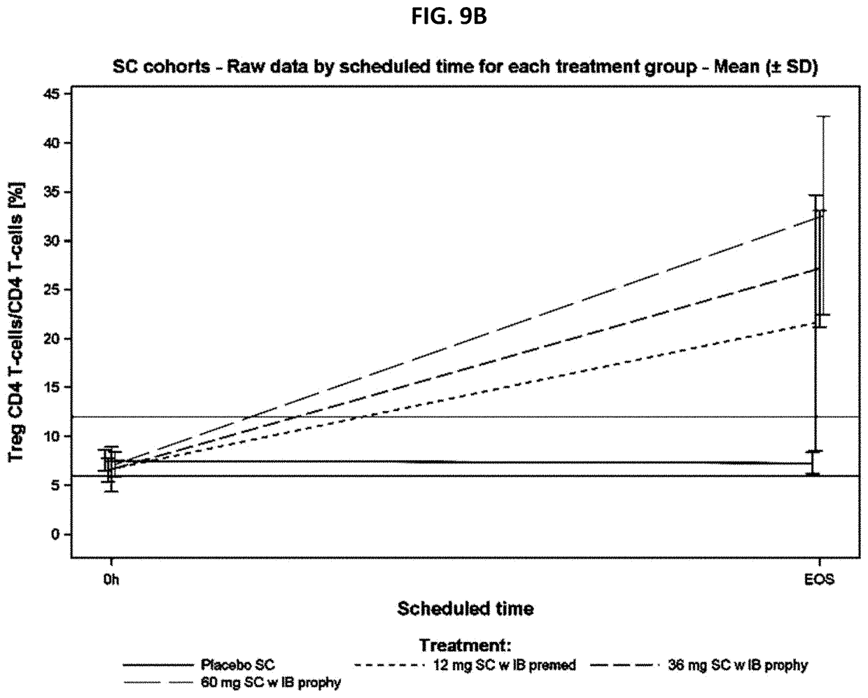

CD4⁺ cells with a phenotype consistent with regulatory T cells (Treg) were assessed at baseline and EOS. Treg cells are a subset of immunologically important cells implicated in pathogenesis of MS. Consistent with other T-cell subsets, Treg cell counts were depleted in all AB1 groups in a dose-dependent manner. However, as a percentage of CD4⁺ cells, Treg cells were increased at EOS compared with baseline in all IV groups in a dose-dependent manner (FIG. 9A).

(ii) SC Administration

Dose-dependent lymphocyte depletion was observed in all AB1 SC groups as shown in FIG. 3B. In the lowest SC dose group (12 mg; n=6), the mean lymphocyte count was reduced from 2.459/nL at baseline to a nadir of 0.566/nL at day 4 post treatment. The lymphocyte count was still below baseline at EOS (0.779/nL). In the highest SC dose group (60 mg; n=6), the mean count was reduced from 2.484/nL at baseline to a nadir of 0.114/nL at 4 days post treatment, and remained substantially below baseline at EOS (0.286/nL). The lowest individual patient values were 0.05/nL, observed at several time points in the 60 mg SC group. Depletion across all lymphocyte subpopulations, including T cells, B cells, NK cells, and various subsets thereof, was observed in all groups receiving AB1 by SC injection.

All lymphocyte subsets showed generally similar temporal profiles. The timing of nadir in SC groups was variable, occurring from 6 to 48 hours post treatment; maximal depletion was not consistently attained until 48 hours, and generally occurred sooner in higher dose cohorts. Subsequent gradual and only partial recovery of cell counts through EOS was observed in most cases. Lymphocyte repopulation began earlier in lower dose groups that showed less complete depletion, but patients generally did not fully recover to baseline values for T or B cells by EOS.

pDC counts remained stable following SC AB1 and were comparable to placebo (FIG. 4B). NK cell counts showed marked depletion following SC AB1 versus placebo, but recovered by Day 10 (FIG. 5B). CD4⁺, CD8⁺, and CD19⁺ lymphocyte counts decreased dose-dependently and less rapidly with SC versus IV administration (FIGS. 6B, 7B, and 8B). Mean counts were >90% lower than baseline at the end of study in the two highest SC dose cohorts.

Treg cells were assessed at baseline and EOS. Consistent with other T-cell subsets, Treg cell counts were depleted in all SC AB1 groups in a dose-dependent manner. However, as a percentage of CD4⁺ cells, Treg cells were increased at EOS compared with baseline in all SC groups in a dose-dependent manner (FIG. 9B). The maximal mean increase was observed in the highest dose groups (60 mg; n=6), rising from 7.131% at baseline to 32.559% at EOS (month 1). For comparison, the Treg percentage at the same time points in Lemtrada® studies was 3.59% at baseline and increased to 12.44% at month 1.

In sum, this Phase 1b study established the safety of AB1 in doses up to 60 mg SC. The maximum IV dose of 12 mg and the SC doses of 36 and 60 mg did not elicit any severe or serious AEs and achieved the desired pharmacodynamic effect of sustained lymphocyte depletion. This study as well as preclinical studies in the huCD52 transgenic mouse model demonstrated that AB1 elicited lymphocyte depletion and repopulation similar to that observed for Lemtrada®. Notably, similar alterations in the proportion of key lymphocyte subsets, including an increase in the percentage of Treg, were also observed.

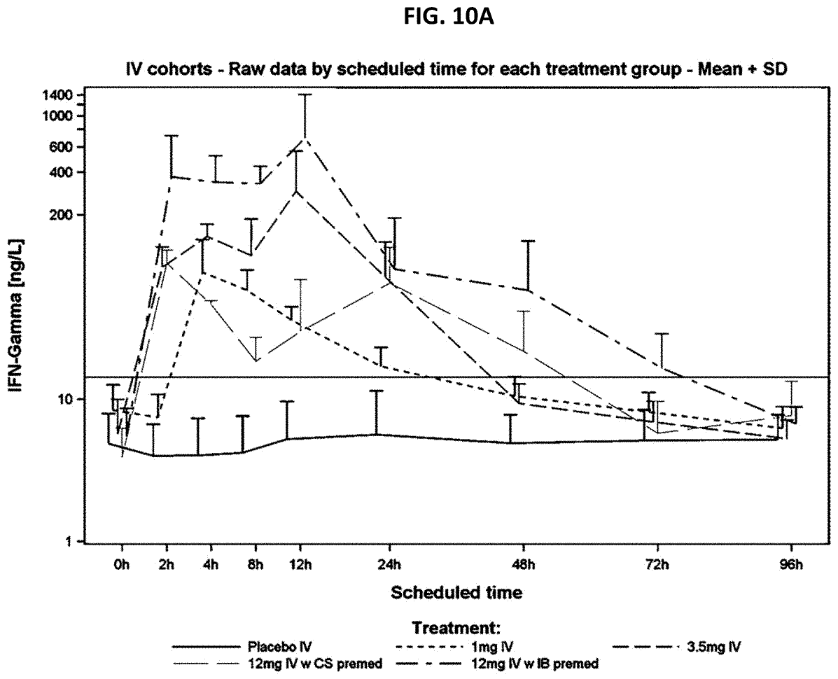

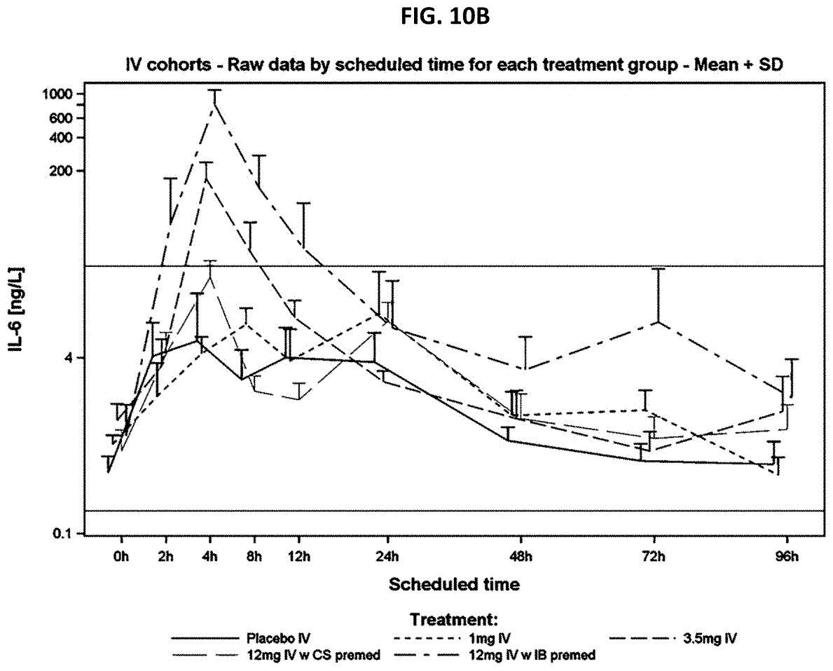

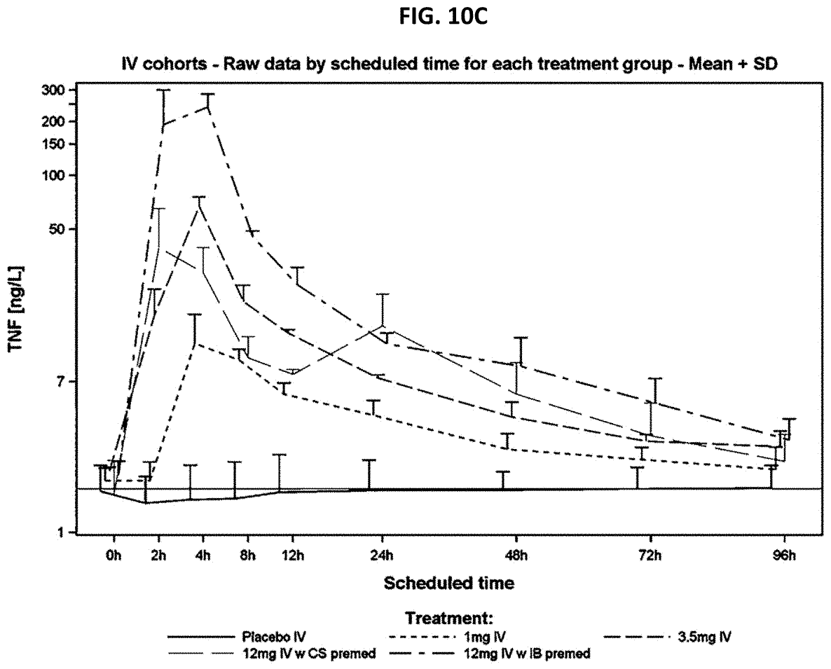

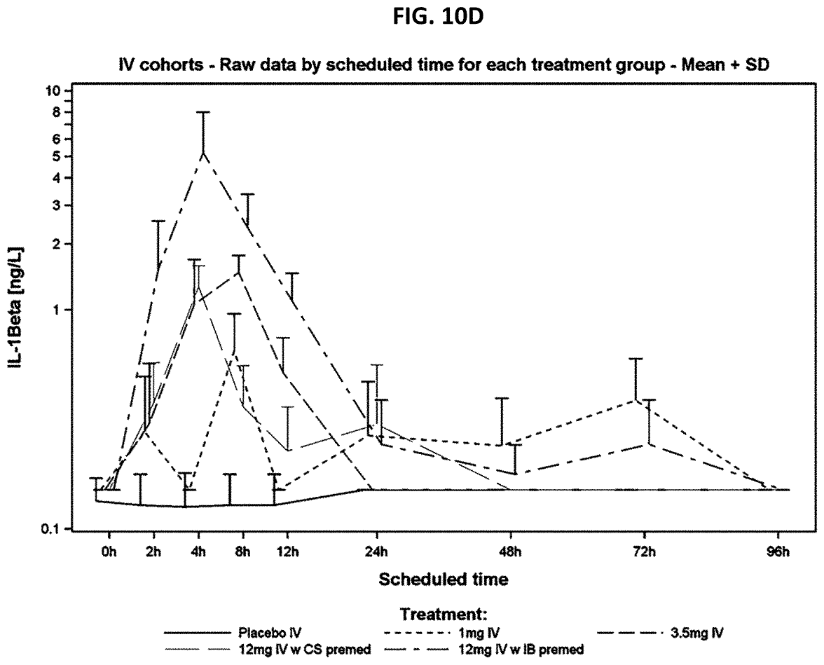

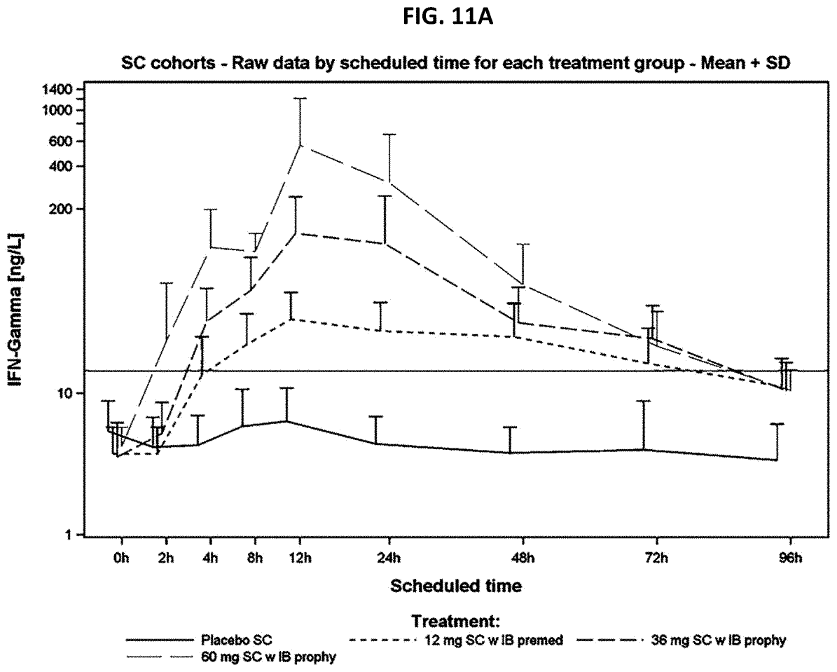

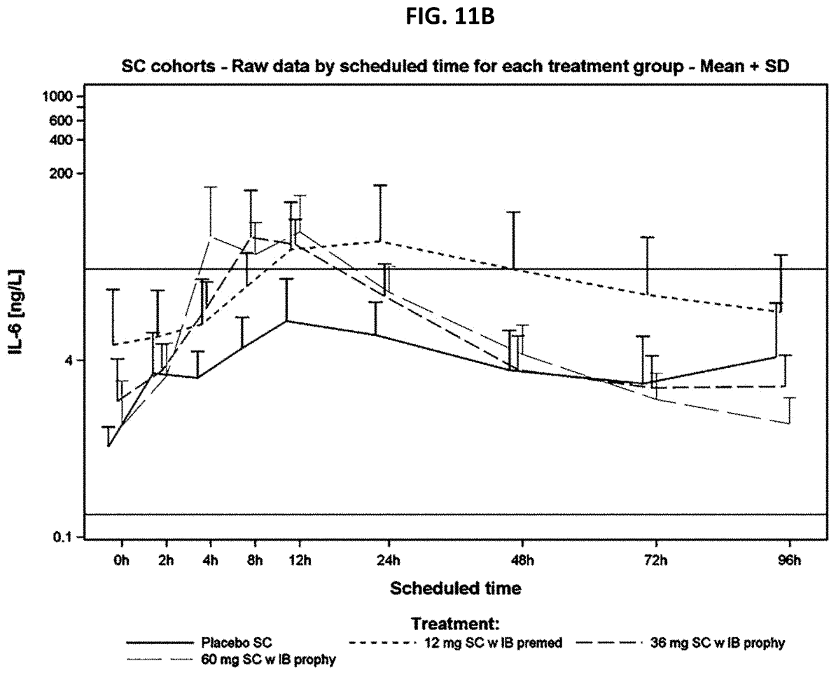

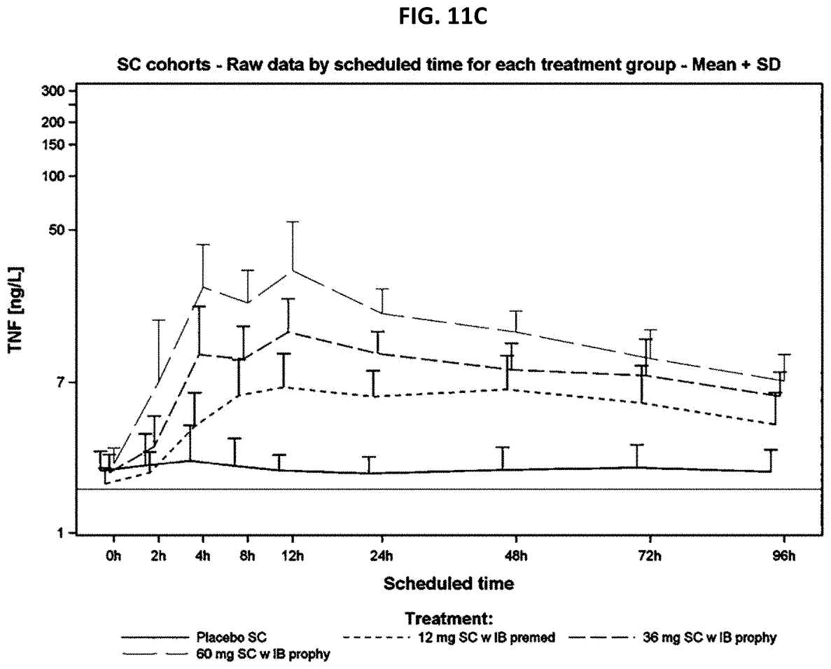

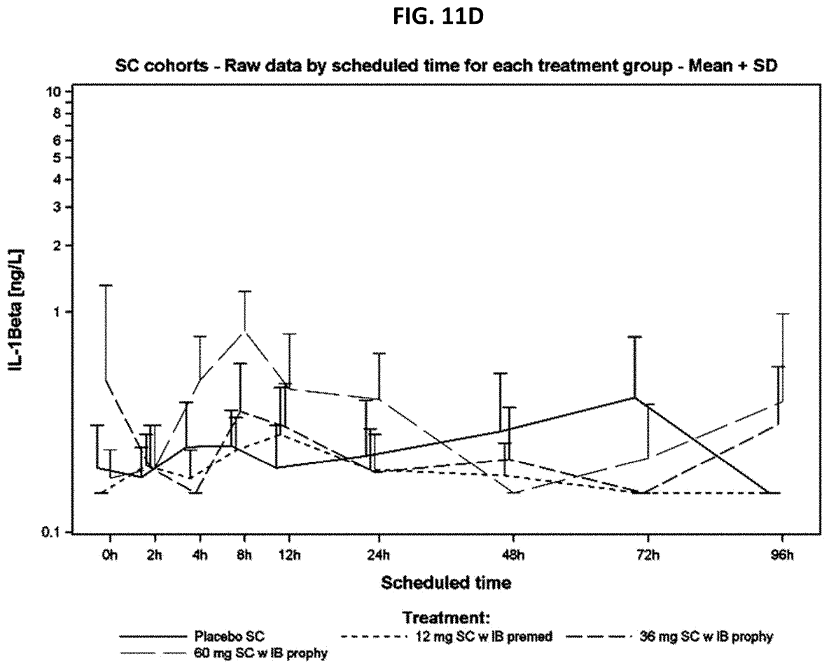

Additionally, following AB1 administration, cytokine increases were observed in INF-γ, IL-6, TNF-α, and IL-1β. For IV, the increases started shortly after the antibody administration, reaching the peaks in four to twelve hours (FIGS. 10A-10D). For SC, the cytokine increases started approximately two hours after the antibody administration, reaching the peaks in four to twelve hours (FIGS. 11A-11D). For both IV and SC, the increases were generally dose-dependent (excluding the 12-mg cohort with steroids) and started to decrease on the same day and normalized by day 3. The mean peak cytokine levels of IL-6, TNFα, and IL-1β were considerably lower in all of the SC groups than in the IV groups at doses that elicited comparable lymphocyte depletion, including the 60-mg SC group with ibuprofen prophylaxis as compared to the 12-mg IV group with ibuprofen pre-medication. The mean maximal levels of INF-γ measured in the highest doses of each IV and SC administration were similar. The overall data indicate that AB1 SC treatment will have improved safety and tolerability and reduced immunogenicity.

Compared to IV administration of Lemtrada®, SC administration of AB1 will be more convenient and cost-effective (SC vs. IV; a single dose vs. multi-day infusion). Further, as evidenced by the low severity of IARs in the absence of steroid premedication, the AB1 SC treatment will have reduced immunogenicity potential and improved safety and tolerability. Since lymphocyte depletion was incomplete in some patients at 12 mg SC and was delayed in some patients at 36 mg SC relative to patients at 60 mg SC, 60 mg SC may be a preferred dosage to ensure lymphocyte depletion in all patients and to achieve the best therapeutic effect.

### Example 5: Pharmacokinetic and Pharmacodynamic Modeling to Support Dose Selection

A population pharmacokinetic (PK)/pharmacodynamic (PD) model was developed to characterize the relationship between AB1 exposure and T lymphocyte depletion and repopulation in MS patients, and to perform clinical trial simulations (CTS) to support dose and dose regimen selection.

A. Population Pharmacokinetics

A population pharmacokinetic (popPK) model was developed using pooled data from 33 patients with progressive MS from the above study after AB1 IV or SC administration. Each patient provided a total of 15 samples for PK analysis: at pre-dose; and at 2, 4, 8, 24, 36, 48, 72, 96, 144, 216, 336, 672, 1416, and 2136 hours after IV or SC administration.

The population PK analysis was conducted in Monolix version 4.4 implementing the Stochastic Approximation Expectation Maximization (SAEM) algorithm. The PK of AB1 was best described by a 2-compartment model with first order absorption and linear elimination. The inter-individual variability (IIV) on selected PK parameters was described by an exponential model and the residual error was described by the combination of additive and proportional error models.

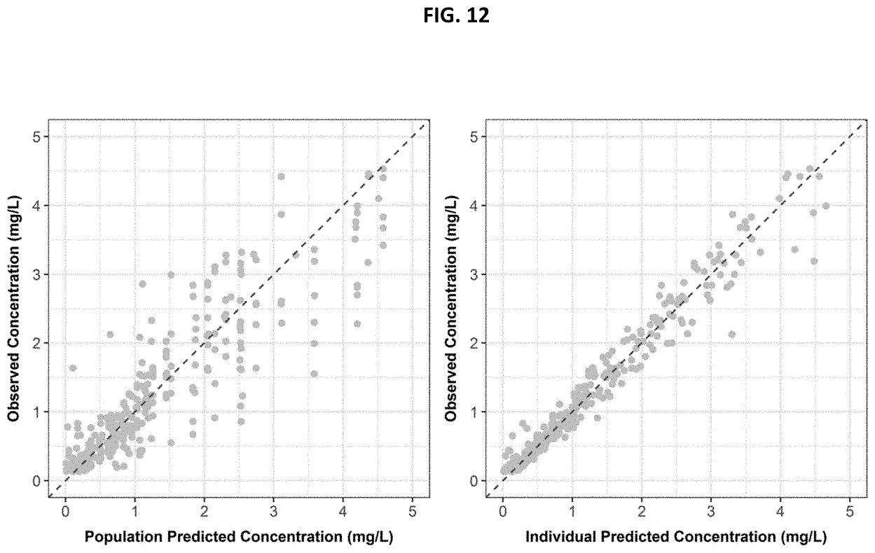

The model parameter estimates are summarized in Table 2. The model estimated that AB1 typical clearance (CL) and steady-state volume of distribution are 0.62 L/day (27.5 mL/hr) and 8.96 L, respectively. These estimates are consistent with the reported PK properties of AB1 as described above. The typical bioavailability of AB1 was fixed to the reported value of 1 as described above. The PK parameters were estimated with good precision (% relative standard error [RSE]<30%) and the IIV was modest for CL and central volume of distribution (Vc) (26% coefficient of variation (CV) and 30% CV, respectively). The adequacy of the popPK model was further demonstrated by the goodness-of-fit plot as shown in FIG. 12. Collectively, the popPK model well characterized AB1 exposure in patients with progressive MS after SC or IV administration.

TABLE 2
Parameter Estimates of AB1 Population PK Model

| | | Population | | Inter-individual | |
|---|---|---|---|---|---|
| Parameter | | Mean | | Variability | |
| (unit) | Definition | Estimate | % RSE | Estimate | % RSE |
| Ka (1/day) | Absorption rate constant | 0.56 | 15 | 0.61 | 17 |
| CL (L/day) | Clearance | 0.62 | 6 | 0.26 | 20 |
| Vc (L) | Central volume of | 4.86 | 8 | 0.30 | 19 |
| | distribution | | | | |
| Vp (L) | Peripheral volume of | 4.10 | 10 | 0.34 | 25 |
| | distribution | | | | |
| Q (L/day) | Distribution clearance | 3.39 | 10 | — | — |
| F | Bioavailability | 1 | Fixed | — | — |
| Vss (L) | Total volume of | 8.96 | — | — | — |
| | distribution | | | | |
| σ1 | Proportional residue error | 0.11 | 10 | — | — |
| σ2 | Additive residue error | 0.07 | 15 | — | — |

B. Pharmacokinetics/Pharmacodynamics Relationships

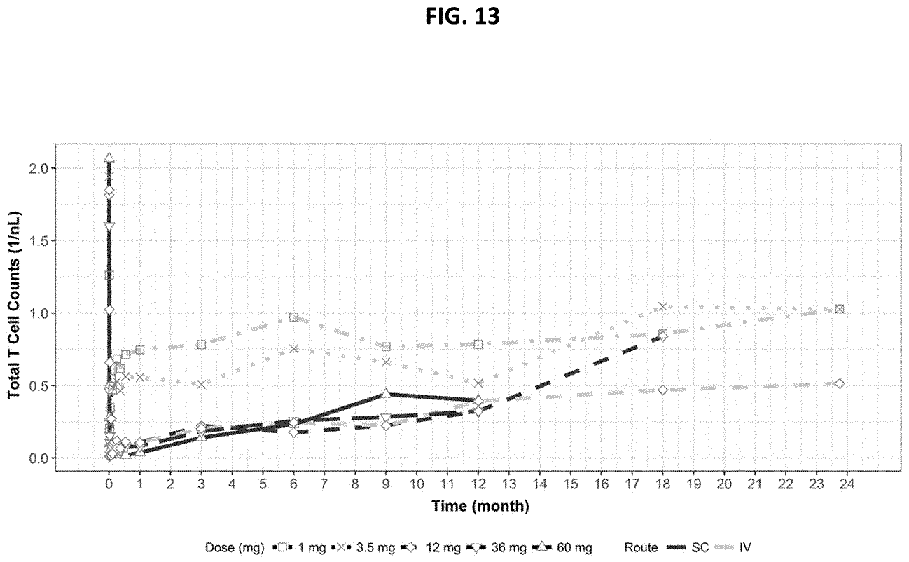

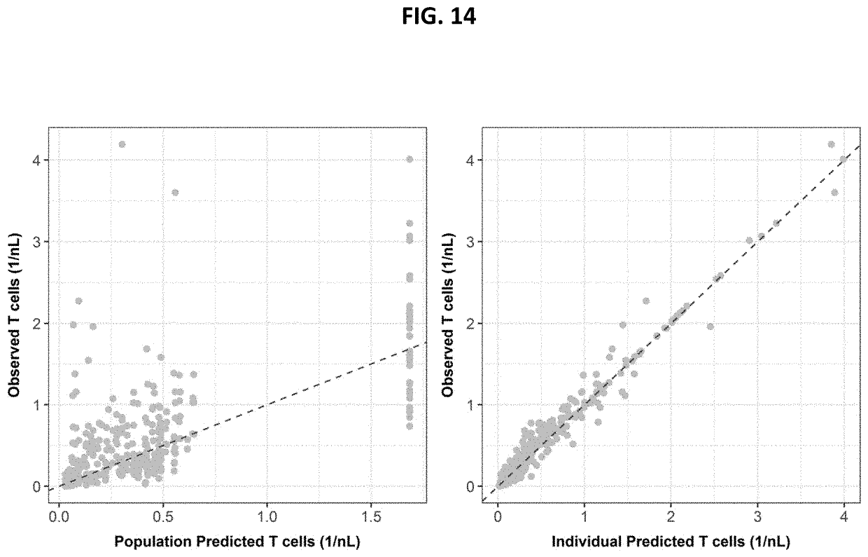

The exposure-response (T lymphocytes) relationship following AB1 treatment in the patients was evaluated by graphical exploratory analysis as well as population PK/PD modeling. The median T cell depletion and repopulation profile following AB1 treatment is provided in FIG. 13. After administration, AB1 elicited rapid and long-lasting depletion of circulating T lymphocytes, followed by a slow repopulation phase. The maximum extent of T lymphocyte depletion was dose-dependent and very substantial in all AB1 dose groups, confirming the desired biological activity of AB1. The median absolute T lymphocyte counts decreased more than 90% from baseline after a 12 mg IV dose as well as after 36 and 60 mg SC doses. T lymphocyte recovery began earlier at lower doses that showed incomplete depletion. The median absolute T lymphocyte counts decreased from baseline at month 12 were still >80% at 60 mg SC and were close to 80% at 36 mg SC and 12 mg IV doses.

C. Mechanism-Based PK/PD Model for AB1 Treatment Effects on T Lymphocytes

A mechanism-based PK/PD model with direct and indirect treatment effects on T lymphocyte dynamics (depletion and repopulation) was developed using pooled data from the patients. The T lymphocytes for PK/PD analysis were collected at pre-dose; at 6, 12, 24, 48, and 72 hours; at 7, 10, and 15 days; and at 1, 3, 6, 9, 12, 18 and 24 months after AB1 IV or SC administration. The PK/PD analysis was sequentially conducted in Monolix version 4.4 implementing the SAEM algorithm. This model was employed to describe physiological homeostasis of T lymphocyte dynamics, proliferation of precursor T lymphocytes, time-dependent migration, elimination from circulating blood, and feedback regulation.

Following AB1 administration, the depletion of T lymphocytes was directly stimulated by AB1 systemic concentration with an Emax function to mimic the AB1-elicited T cell lysis via either ADCC or CDC. Emax is a measure of the maximum stimulation effect of circulating T cell depletion. Additionally, the migration of T cells into circulating blood was indirectly inhibited by AB1 concentration. The model parameter estimates were summarized in Table 3. In general, the precision for the majority of parameters was high throughout (% RSE<30%). Additionally, the model estimated a T lymphocyte baseline value of 1,680×10⁶/L, which is consistent with the studies described above. The T lymphocyte migration time is estimated as 2.44 days, which is within the literature reported time window of lymphocyte trafficking in humans (Mager et al., J Clin Pharmacol 43:1216-1227 (2003)). Further, the adequacy of the mechanism-based PK/PD model was demonstrated by the goodness-of-fit plot as shown in FIG. 14. Collectively, the exposure-response relationship of T lymphocytes in response to AB1 treatment from patients with progressive MS after SC or IV administration was well characterized by the PK/PD model.

TABLE 3
Parameter Estimates of AB1 Population PK/PD Model

| | | Population | | Inter-individual | |
|---|---|---|---|---|---|
| Parameter | | Mean | | Variability | |
| (unit) | Definition | Estimate | % RSE | Estimate | % RSE |
| Base (10⁶/L) | T cells at baseline | 1680 | 7 | 0.41 | 13 |
| MTT (day) | T cell migration transit time | 2.44 | 12 | 0.61 | 15 |
| Emax | Maximum stimulation effect of | 33 | Fixed | 0.56 | 27 |
| | circulating T cell depletion | | | | |
| EC50 (mg/L) | Concentration achieves 50% of | 4.16 | 21 | 1.12 | 14 |
| | circulating T cell depletion | | | | |
| SL | Linear inhibition effect of precursor | 0.0034 | 45 | 1.53 | 26 |
| (1/(mg/L)) | T cell migration | | | | |
| Circ_L | Physiological lower limit of | 10 | 44 | 1.93 | 19 |
| (10⁶/L) | circulating T cells | | | | |
| Kdeg (1/day) | Circulating T cell depletion rate | 7.22 | 12 | 0.57 | 16 |
| | constant | | | | |
| FB | Negative feedback power coefficient | 0.0004 | 30 | 1.1 | 26 |
| | on precursor T cell migration | | | | |
| σ1 | Additive residue error | 0.11 | 5 | — | — |

D. Simulations of AB1 Treatment Effects on T Lymphocytes

To support the dose selection for the first dose of AB1, the mechanism-based PK/PD model was used to conduct CTS to predict the exposure-response relationship for T lymphocytes at different AB1 doses at month 1 over a 1-year treatment period in a virtual MS population. Those results were compared to the T lymphocyte counts observed following administration of IV alemtuzumab at 12 mg/day×5 days (total=60 mg) in two Phase 3 studies in patients with RRMS (CAMMS323 and CAMMS324). The extent of T lymphocyte depletion at month 1 following single SC administration of 12, 24, 36, 48 and 60 mg of AB1 was simulated in 100 trials with 500 virtual MS patients at each dose in each trial.

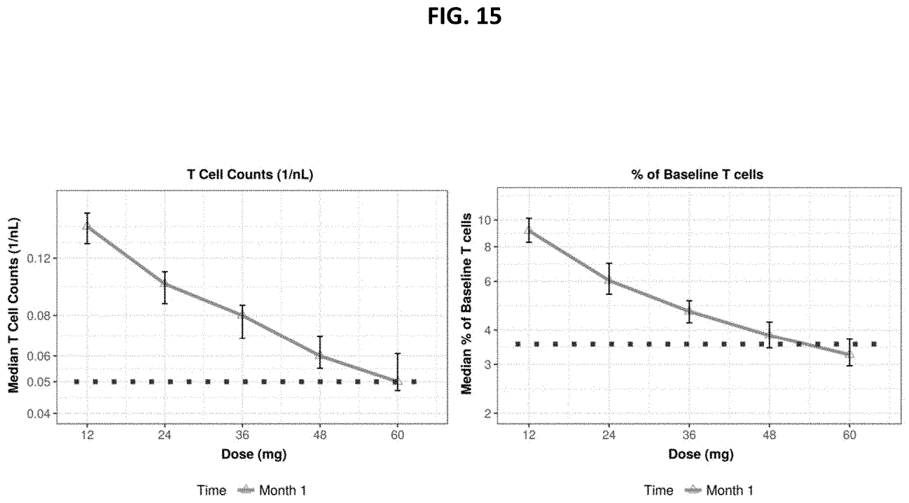

The model-predicted extent of T lymphocyte depletion at month 1 following AB1 SC single dose administration is shown in FIG. 15. Descriptive statistics of the predicted extent of T lymphocyte depletion are summarized in Table 4. Table 4 shows simulation predictions by a population PK/PD model of absolute T lymphocyte counts at month 1 after a first AB1 treatment versus observed counts at month 1 after a first alemtuzumab treatment. Simulations were performed and summarized for 100 replicates of studies (500 patients/treatment/trial).

TABLE 4
Predicted Absolute T Lymphocyte Counts
at Month 1 after 1st AB1 Treatment versus Observed
Counts at Month 1 after 1st Alemtuzumab Treatment

| | T cell counts (×10⁶/L) | | % of Baseline T cells | |
|---|---|---|---|---|
| | Predicted Median | | Predicted Median | |
| | (5^{th} centile- | | (5^{th} centile-95^{th} | |
| Regimen | 95^{th} centile) | Reference^{a} | centile) | Reference^{a} |
| 12 mg SC | 150 | 50 (20, | 9.2 | 3.6 |
| | (130-160) | 130) | (8.3-10.1) | |
| 24 mg SC | 100 | | 6.1 | |
| | (87-110) | | (5.4-7.0) | |
| 36 mg SC | 80 | | 4.7 | |
| | (68-86) | | (4.2-5.1) | |
| 48 mg SC | 60 | | 3.8 | |
| | (55-69) | | (3.4-4.3) | |
| 60 mg SC | 50 | | 3.3 | |
| | (47-61) | | (3.0-3.7) | |

^{a}Median (Q1, Q3) absolute T lymphocyte cell counts in alemtuzumab studies CAMMS323 and CAMMS324 (pooled); administered via 5 daily 12 mg IV infusions during cycle 1 at month 0

In general, AB1 elicited dose-dependent T cell depletion, and the observed T lymphocyte response in two alemtuzumab Phase 3 studies CAMMS323 and CAMMS324 (a median of 50×10⁶/L total T lymphocyte count at month 1) was predicted to be achieved with the 60 mg SC regimen. Additionally, CTS indicated that the AB1 60 mg SC regimen was more effective than other simulated regimens in achieving an extent of T lymphocyte depletion, comparable to the effect of alemtuzumab 60 mg IV, supporting 60 mg SC as the selected first treatment dose for achieving the desired PD effects in MS patients.

The aim for the second SC injection of AB1, given 12 months after the initial injection, is to achieve similar lymphocyte depletion as with the first SC injection. With 60 mg as the AB1 dose for the first treatment, CTS were performed to compare the extent of T lymphocyte depletion one month after the second treatment of 36 mg SC and 60 mg SC in 100 trials with 500 virtual MS patients at each dose in each trial. The results further suggested that using 60 mg for the second treatment would produce a similar extent of T lymphocyte depletion at month 1 compared to that observed with 60 mg IV alemtuzumab administration (Tables 4 and 5).

Table 5 shows simulation prediction by the population PK/PD model of absolute T lymphocyte counts at month 1 after a second AB1 treatment versus observed counts at month 1 after first and second alemtuzumab treatments. Simulations were performed and summarized for 100 replicates of studies (500 patients/treatment/trial).

TABLE 5
Predicted Absolute T Lymphocyte Counts at Month 1 after 2nd AB1
Treatment versus Observed Counts at Month 1 after 2^{nd} Alemtuzumab Treatment

| Treatment | T cell counts (10⁶/L) | | | % of Baseline T cells | |
|---|---|---|---|---|---|
| (first | Predicted | | Observed | | |
| treatment/ | Median | Observed | (alemtuzumab | Predicted Median | |
| second | (5^{th} centile-95^{th} | (alemtuzumab | second | (5^{th} centile-95^{th} | |
| treatment) | centile) | first course^{a}) | course^{b}) | centile) | Reference^{b} |
| 60 mg SC/ | 70 | 50 | 80 | 27.8 | 16.3 |
| 36 mg SC | (63-83) | | | (24.9-30.0) | |
| 60 mg SC/ | 50 | | | 20.0 | |
| 60 mg SC | (47-60) | | | (18.0-22.2) | |

^{a}Median absolute T lymphocyte cell counts in alemtuzumab studies CAMMS323 and CAMMS324 (pooled); 60 mg IV administered via 5 daily 12 mg IV infusions during course 1 at month 0
^{b}Median absolute T lymphocyte cell counts in alemtuzumab studies CAMMS323 and CAMMS324 (pooled); 36 mg IV administered via 3 daily 12 mg IV infusions during course 2 at month 12

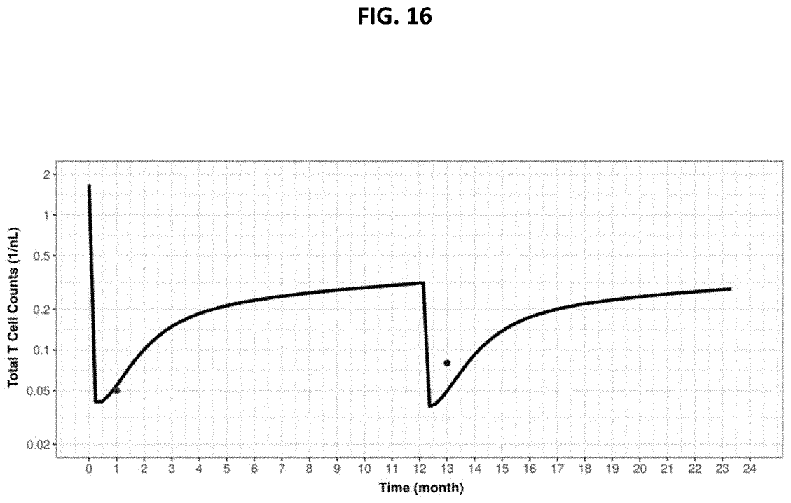

The PD response to two annual treatments with 60 mg SC AB1 predicted by CTS was compared with the response to the standard alemtuzumab treatment regimen in FIG. 16. The simulation data show that 60 mg AB1 SC injection as the first and second treatment will deplete T lymphocytes similarly with each treatment, and that depletion is more complete than that observed after the second alemtuzumab treatment which consists of 3 daily IV infusions of 12 mg each (total=36 mg).

### Example 6: Treatment of Relapsing MS with SC AB1

This example describes a treatment regimen for RMS patients by administration (by a health care provider or self-administration under the supervision of a health care provider) of 60 mg of AB1 via a single SC dose, followed by another single SC dose of 60 mg one year later and then any as-needed retreatment in subsequent years. The treatment regimen may include acyclovir (e.g., 200 mg acyclovir PO twice daily for 28 days beginning on the first day of each antibody treatment course). Additionally or alternatively, the treatment regimen may include methylprednisolone.

The term “RMS” includes patients with RRMS, SPMS with relapses, and a clinically isolated demyelination event with evidence of dissemination of lesions in time and space on the MRI (see, e.g., European Medicines Agency, Committee for Medicinal Products for Human Use's “Guideline on Clinical Investigation of Medicinal Products for the Treatment of Multiple Sclerosis” (Rev. 2, 2015)). Prevention and/or modification of relapse features as well as prevention or delay of the accumulation of disability due to relapses are meaningful goals of treatment in RMS. Disease conditions are evaluated by well-established criteria, such as the Expanded Disability Status Scale (EDSS), MS Functional Composite (MSFC), and MRI. It is expected that treatment of RMS with two doses of 60 mg SC, one year apart, will achieve both goals and that the efficacy of this treatment will be comparable or superior to treatment with, e.g., 14 mg QD PO of teriflunomide (Aubagio®) or Lemtrada®. The efficacy of the treatment is indicated by clinical endpoints of, for example, (1) a reduction (e.g., 45% or more) in annualized relapse rate (ARR) in a population of patients (e.g., 450 or more) assuming ARR in the control group is 0.29 to 0.49 (alpha=0.05, 2-sided) (e.g., an increase in the proportion of relapse-free patients by 24 months); (2) a risk reduction (e.g., 35% or more) on 3- or 6-month confirmed disability worsening (CDW) as assessed by EDSS in a population of patients (e.g., 900 or more) assuming a 15% to 20% event rate by year 2 in the control group (alpha=0.05, 2-sided); (3) a reduction in the development of new or enlarging T1- and/or T2-hyperintense lesions as detected by brain MRI (e.g., at months 6, 12, and 24); (4) an increase in confirmed disability improvement (CDI) over at least six months; (5) a reduction in brain volume loss (e.g., as detected by brain MRI from baseline or month 6 to month 24); (6) an improvement in EDSS from baseline to month 12 and/or 24; (7) an improvement in timed 25 foot walk from baseline to month 12 and/or 24; (8) an improvement in 9-hole peg test from baseline to month 12 and/or 24; (9) an improvement in EDSS-Plus composite score from baseline to month 24; (10) a reduction in proportion of patients diagnosed with RRMS at enrollment who have progressed to SPMS at end of study; (11) an increase in the proportion of patients who have no evidence of disease activity (NEDA) by month 24; (12) an increase in low contrast visual acuity from baseline to month 12 and/or 24; (13) an increased time to onset of sustained 20% increase in 9-hole peg test for at least 12 weeks; and/or (14) an increased time to onset of sustained 20% increase in timed 25 foot walk for at least 12 weeks. Any combination of these endpoints (e.g., (1) alone, or in combination with any of the other endpoints) may indicate efficacy of the treatment.

After the two annual SC doses of AB1, a patient may be re-treated with another SC dose of AB1 as needed, for example, when the patient displays renewed MS activity. For example, re-treatment may be indicated if: (1) within the previous year, the patient has experienced one or more relapses, or (2) since their last MRI, the patient has accumulated two or more unique lesions on brain or spinal cord MRIs comprising gadolinium-enhancing lesions (e.g., at least 3 mm in any dimension) and/or new or enlarging MRI T2 lesions (e.g., at least 3 mm in any dimension, or showing at least a 3 mm increase).

After each dose of AB1, the patients are monitored for development of any secondary autoimmunity as described above.

### Example 7: Treatment of Primary Progressive MS with SC AB1

This example describes a treatment regimen for primary progressive MS patients by administration (by a health care provider or self-administration under the supervision of a health care provider) of 60 mg of AB1 via a single SC dose, followed by another SC dose of 60 mg one year later and then any as-needed retreatment in subsequent years. The treatment regimen may include acyclovir (e.g., 200 mg acyclovir PO twice daily for 28 days beginning on the first day of each treatment course). Additionally or alternatively, the treatment regimen may include methylprednisolone. It is expected that the treatment will benefit the patients by reducing neurodegeneration and neuroinflammation.

Disease conditions are evaluated by well-established criteria, such as EDSS, MSFC, and MRI. The efficacy of the treatment can be indicated by clinical endpoints of, for example, (1) an increased time to confirmed disability progression, or a risk reduction (e.g., 25% or more) on 6-month CDW, as assessed by EDSS-Plus Composite measure (EDSS, Timed 25-foot walk (T25FW), 9 Hole-Peg Test (9-HPT)) in a population of patients (e.g., 800 or more). Secondary clinical endpoints may include, e.g., (2) time to confirmed disability worsening (confirmed over, e.g., at least three months) as assessed by EDSS-Plus Composite measure; (3) total number of new and/or enlarging T2 hyperintense lesions as detected by brain MRI at, e.g., months 6, 12, and 24; (4) change in brain volume as detected by brain MRI, e.g., from month 6 to month 24; (5) proportion of patients with confirmed disability improvement (CDI) confirmed over, e.g., six months; and (6) annualized relapse rate. Still other endpoints may include, e.g., (7) time to onset of confirmed disability worsening (e.g., 3 or 6 month) as assessed by EDSS; (8) time to onset of confirmed disability worsening (e.g., 3 or 6 month) as assessed by 9HPT; (9) change of EDSS from baseline, e.g., at months 12 and/or 24; (10) change of T25FW test from baseline, e.g., at months 12 and/or 24; (11) change in performance in 9-HPT from baseline to, e.g., months 12 and/or 24; (12) total number of Gd-enhancing T1 hyperintense lesions as detected by brain MRI at, e.g., months 6, 12, and 24; (13) change in brain volume as detected by brain MRI from baseline to, e.g., month 24; (14) change in total T2 lesion volume as detected by brain MRI from baseline to, e.g., month 24; and (15) patient reported outcomes. Any combination of these endpoints (e.g., (1) alone, or in combination with any or all of (2)-(6), and/or in combination with any or all of (7)-(15)) may indicate efficacy of the treatment.

After the two annual SC doses of AB1, a patient may be re-retreated with another SC dose of AB1 as needed, for example, when the patient displays renewed MS activity. For example, re-treatment may be indicated if: (1) within the previous year, the patient has experienced confirmed disability worsening of ≥1 point confirmed over 3 months, or more in patients with a screening EDSS score of <6.0; (2) within the previous year, the patient has experienced confirmed disability worsening of ≥0.5 points confirmed over 3 months, or more in patients with a screening EDSS score of ≥6.0; (3) within the previous year, the patient has experienced one or more relapses; and/or (4) since their last MRI, the patient has accumulated two or more unique lesions on brain or spinal cord MRIs comprising gadolinium-enhancing lesions (e.g., at least 3 mm in any dimension) and/or new or enlarging MRI T2 lesions (e.g., at least 3 mm in any dimension, or showing at least a 3 mm increase).

After each dose of AB1, the patients are monitored for development of any secondary autoimmunity as described above.

### Example 8: Treatment of Secondary Progressive MS with SC AB1

This example describes a treatment regimen for secondary progressive MS patients by administration (by a health care provider or self-administration under the supervision of a health care provider) of 60 mg of AB1 via a single SC dose, followed by another SC dose of 60 mg one year later and then any as-needed retreatment in subsequent years. The treatment regimen may include acyclovir (e.g., 200 mg acyclovir PO twice daily for 28 days beginning on the first day of each treatment course). Additionally or alternatively, the treatment regimen may include methylprednisolone. It is expected that the treatment will benefit the patients by reducing neurodegeneration and neuroinflammation.

Disease conditions are evaluated by well-established criteria, such as EDSS, MSFC, and MRI. The efficacy of the treatment can be indicated by clinical endpoints of, for example, (1) an increased time to confirmed disability progression, or a risk reduction (e.g., 25% or more) on 6-month CDW, as assessed by EDSS-Plus Composite measure (EDSS, Timed 25-foot walk (T25FW), 9 Hole-Peg Test (9-HPT)) in a population of patients (e.g., 800 or more). Secondary clinical endpoints may include, e.g., (2) annualized relapse rate; (3) time to confirmed disability worsening (CDW) (confirmed over at least 3 months) as assessed by EDSS-Plus composite; (4) total number of new and/or enlarging T2 hyperintense lesions as detected by brain MRI at, e.g., months 6, 12, and 24; (5) change in brain volume as detected by brain MRI from, e.g., month 6 to month 24; and (6) proportion of patients with confirmed disability improvement (CDI) confirmed over six months. Still other endpoints may include, e.g., (7) time to onset of confirmed disability progression (e.g., 3 or 6 month) as assessed by EDSS; (8) time to onset of confirmed disability progression (e.g., 3 or 6 month) as assessed by T25FW test; (9) time to onset of confirmed disability progression (e.g., 3 or 6 month) as assessed by 9HPT; (10) change of EDSS from baseline, e.g., at months 12 and/or 24; (11) change of T25FW test from baseline, e.g., at months 12 and/or 24; (12) change in performance in 9-HPT from baseline to, e.g., months 12 and/or 24; (13) proportion of patients with no evidence of disease activity (NEDA); (14) total number of Gd-enhancing T1 hyperintense lesions as detected by brain MRI at, e.g., months 6, 12, and 24; (15) change in brain volume as detected by brain MRI from baseline to, e.g., month 24; (16) change in total T2 lesion volume as detected by brain MRI from baseline to, e.g., month 24; and (17) patient reported outcomes. Any combination of these endpoints (e.g., (1) alone, or in combination with any or all of (2)-(6), and/or in combination with any or all of (7)-(17)) may indicate efficacy of the treatment.

After the two annual SC doses of AB1, a patient may be re-retreated with another SC dose of AB1 as needed, for example, when the patient displays renewed MS activity. For example, re-treatment may be indicated if: (1) within the previous year, the patient has experienced confirmed disability worsening of ≥1 point confirmed over 3 months, or more in patients with a screening EDSS score of <6.0; (2) within the previous year, the patient has experienced confirmed disability worsening of ≥0.5 points confirmed over 3 months, or more in patients with a screening EDSS score of ≥6.0; (3) within the previous year, the patient has experienced one or more relapses; and/or (4) since their last MRI, the patient has accumulated two or more unique lesions on brain or spinal cord MRIs comprising gadolinium-enhancing lesions (e.g., at least 3 mm in any dimension) and/or new or enlarging MRI T2 lesions (e.g., at least 3 mm in any dimension, or showing at least a 3 mm increase).

After each dose of AB1, the patients are monitored for development of any secondary autoimmunity as described above.

1. A method of treating multiple sclerosis (MS) in a human patient in need thereof, comprising:
administering to the patient a humanized monoclonal anti-human CD52 IgG₁ antibody whose heavy chain CDR1-3 and light chain CDR1-3 comprise the amino acid sequences of SEQ ID NOs:5-10, respectively, at a first dose of 12-60 mg, and
after an interval of 12 or more months, administering to the patient the antibody at a second dose of 12-60 mg.

2. The method of claim 1, wherein the antibody comprises a heavy chain variable domain and a light chain variable domain having the amino acid sequences of SEQ ID NOs:3 and 4, respectively.

3. The method of claim 2, wherein the antibody comprises a heavy chain and a light chain having the amino acid sequences of SEQ ID NOs:1 and 2, respectively.

4. The method of claim 1, wherein the patient has relapsing multiple sclerosis (RMS), secondary progressive multiple sclerosis (SPMS), or primary progressive multiple sclerosis (PPMS).

5-6. (canceled)

7. The method of claim 1, wherein the antibody is administered to the patient by intravenous infusion.

8. The method of claim 7, wherein;
a) the first dose is 60 mg administered to the patient over 1-5 days, and the second dose is 36 mg administered to the patient over 1-3 days;
b) the first dose is administered to the patient at 12 mg/day for 5 days, and the second dose is administered to the patient at 12 mg/day for 3 days;
e) each of the first and second doses is 48 mg administered to the patient over 1-4 days; or
d) each of the first and second doses is administered to the patient at 12 mg/day for 4 days.

9-11. (canceled)

12. The method of claim 1, wherein the antibody is administered to the patient by subcutaneous injection.

13. The method of claim 12, wherein;
a) the first and second doses are both 60 mg;
b) the first dose is 60 mg and the second dose is 36 mg;
e) the first and second doses are both 36 mg; or
d) the first and second doses are both 48 mg.

14-16. (canceled)

17. The method of claim 12, wherein each dose is administered in a single injection.

18. The method of claim 1, wherein the patient is medicated with a corticosteroid, an antihistamine, an antipyretic, or an NSAID before, during, and/or after each administering step.

19. The method of claim 18, wherein;
a) the patient is medicated with methylprednisolone before and/or after each administering step;
b) the patient is medicated with ibuprofen before and/or after each administering step: or
c) the patient is medicated with naproxen before and/or after each administering step.

20-21. (canceled)

22. The method of claim 1, further comprising administering to the patient another dose of the antibody at a dose of 12-60 mg if the patient displays renewed MS activity or worsening of the disease.

23. A method of treating multiple sclerosis (MS) in a human patient in need thereof, comprising:
administering to the patient by subcutaneous injection an anti-human CD52 antibody whose heavy chain and light chain comprise the amino acid sequences of SEQ ID NOs:1 and 2, respectively, at a first dose of 60 mg, and
at 12 months, administering to the patient by subcutaneous injection the antibody at a second dose of 60 mg.

24. The method of claim 23, wherein the patient has RMS, SPMS, or PPMS.

25. The method of claim 23, wherein each of the first and second doses is administered in a single injection.

26. The method of claim 23, wherein the patient is treated with a corticosteroid, an antihistamine, an antipyretic, or an NSAID per os before and/or after each of the administering steps.

27. The method of claim 23, wherein the patient is treated with acyclovir per os.

28. The method of claim 27, wherein the acyclovir is administered to the patient at 200 mg twice daily for 28 days beginning on the first day of each antibody treatment course.

29. The method of claim 23, wherein the patient is treated with methylprednisolone per os.

30-35. (canceled)

36. A kit comprising:
a) a container that comprises a single dose of 12-60 mg of a humanized monoclonal anti-human CD52 IgG₁ antibody whose heavy chain CDR1-3 and light chain CDR1-3 comprise the amino acid sequences of SEQ ID NOs:5-10, respectively, wherein the container is for subcutaneous delivery; and
b) a label associated with the container.

37-39. (canceled)


SEQUENCE LISTING (11 sequences)

SEQ ID NO: 1 - protein, 444 aa, synthetic construct
  REGION 1..444: note = source = /note="Description of Artificial Sequence: Syntheticpolypeptide"
EVQLVESGGGLVQPGGSLRLSCAASGFPFSNYWMNWVRQAPGKGLEWVGQIRLKSNNYATHYAESVKGRFTISRDDSKNSLYLQMNSLKTEDTAVYYCTPIDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 218 aa, synthetic construct
  REGION 1..218: note = source = /note="Description of Artificial Sequence: Syntheticpolypeptide"
DIVMTQTPLSLSVTPGQPASISCKSSQSLLYSNGKTYLNWVLQKPGQSPQRLIYLVSKLDSGVPDRFSGSGSGTDFTLKISRVEAEDVGVYYCVQGSHFHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 114 aa, synthetic construct
  REGION 1..114: note = source = /note="Description of Artificial Sequence: Syntheticpolypeptide"
EVQLVESGGGLVQPGGSLRLSCAASGFPFSNYWMNWVRQAPGKGLEWVGQIRLKSNNYATHYAESVKGRFTISRDDSKNSLYLQMNSLKTEDTAVYYCTPIDYWGQGTTVTVSS

SEQ ID NO: 4 - protein, 111 aa, synthetic construct
  REGION 1..111: note = source = /note="Description of Artificial Sequence: Syntheticpolypeptide"
DIVMTQTPLSLSVTPGQPASISCKSSQSLLYSNGKTYLNWVLQKPGQSPQRLIYLVSKLDSGVPDRFSGSGSGTDFTLKISRVEAEDVGVYYCVQGSHFHTFGQGTKLEIK

SEQ ID NO: 5 - protein, 10 aa, synthetic construct
  REGION 1..10: note = source = /note="Description of Artificial Sequence: Syntheticpeptide"
GFPFSNYWMN

SEQ ID NO: 6 - protein, 19 aa, synthetic construct
  REGION 1..19: note = source = /note="Description of Artificial Sequence: Syntheticpeptide"
QIRLKSNNYATHYAESVKG

SEQ ID NO: 7 - protein, 5 aa, synthetic construct
  REGION 1..5: note = source = /note="Description of Artificial Sequence: Syntheticpeptide"
TPIDY

SEQ ID NO: 8 - protein, 16 aa, synthetic construct
  REGION 1..16: note = source = /note="Description of Artificial Sequence: Syntheticpeptide"
KSSQSLLYSNGKTYLN

SEQ ID NO: 9 - protein, 7 aa, synthetic construct
  REGION 1..7: note = source = /note="Description of Artificial Sequence: Syntheticpeptide"
LVSKLDS

SEQ ID NO: 10 - protein, 8 aa, synthetic construct
  REGION 1..8: note = source = /note="Description of Artificial Sequence: Syntheticpeptide"
VQGSHFHT

SEQ ID NO: 11 - protein, 12 aa, Homo sapiens
GQNDTSQTSSPS